(19) United States
(12) Patent Application Publication
(10) Pub. No.: US 2014/0234322 A1
(43) Pub. Date: Aug. 21, 2014

(54) THROMBOPOIETIN MIMETICS FOR THE TREATMENT OF RADIATION OR CHEMICAL INDUCED BONE MARROW INJURY

(71) Applicants: Yuhchyau CHEN, Pittsford, NY (US); J.H. David WU, Pittsford, NY (US); Jane L. LIESVELD, Rochester, NY (US)

(72) Inventors: Yuhchyau CHEN, Pittsford, NY (US); J.H. David WU, Pittsford, NY (US); Jane L. LIESVELD, Rochester, NY (US)

(73) Assignee: UNIVERSITY OF ROCHESTER, Rochester, NY (US)

(21) Appl. No.: 14/268,126

(22) Filed: May 2, 2014

Related U.S. Application Data
(62) Division of application No. 13/838,111, filed on Mar. 15, 2013.
(60) Provisional application No. 61/682,544, filed on Aug. 13, 2012.
(60) Provisional application No. 61/728,465, filed on Nov. 20, 2012.

(51) Int. Cl. A61K 31/4152; A61K 45/06; A61K 38/19; C07K 16/28; A61K 39/395

(52) U.S. Cl. CPC A61K 31/4152; C07K 16/2866; A61K 39/3955; A61K 38/196; A61K 45/06
USPC 424/139.1; 514/404; 514/7.8

(57) ABSTRACT

Disclosed are transgenic non-human mammals, which useful for the screening of thrombopoietin mimetics, thrombopoietin receptor agonists, or thrombopoietin receptor antagonists active on the human thrombopoietin receptor. The transgenic non-human mammal has a genome that comprises a stably integrated transgene construct comprising a polynucleotide sequence encoding a humanized thrombopoietin receptor wherein said transgenic non-human mammal has a baseline blood platelet count corresponding to a physiological blood platelet count of a matched non-transgenic non-human mammal. The chimeric thrombopoietin receptor comprises either the transmembrane domain of a human thrombopoietin receptor or both the extracellular and transmembrane domains of a human thrombopoietin receptor operably coupled to a cytoplasmic domain of a non-human thrombopoietin receptor.

This application is a division of U.S. patent application Ser. No. 13/838,111, filed Mar. 15, 2013, which claims the benefit of U.S. Provisional Patent Application Ser. Nos. 61/682,544, filed Aug. 13, 2012 and 61/728,465, filed Nov. 20, 2012, each of which is hereby incorporated by reference in its entirety.

This invention was made with government support under grant HHSO100200800058C from the Biomedical Advanced Research and Development Authority, U.S. Department of Health and Human Services; and grant U19A1067733 from the Center for Medical Countermeasures against Radiation Program, National Institute of Health/National Institute of Allergy and Infectious Disease. The government has certain rights in this invention.

### FIELD OF THE INVENTION

The present invention relates to methods of treating radiation or chemical induced bone marrow injury using thrombopoietin (TPO) mimetic. The present invention also relates to transgenic knock-in animals expressing a humanized TPO receptor and methods for screening TPO mimetics.

### BACKGROUND OF THE INVENTION

The increased threat of terrorism underscores the compelling need to develop improved treatments to protect all segments of the civilian population, and specifically, against radiation injury to hematopoietic systems, which is the most radiosensitive organ system. Clinical manifestations of radiation bone marrow injury such as neutropenia and thrombocytopenia directly impact the survival of exposed victims. Severe neutropenia increases the risk of sepsis and death due to opportunistic infections. Thrombocytopenia increases the risk of hemorrhage and death due to internal and external bleeding. While rhG-CSF reduces death rates from infection and sepsis by promoting the recovery of neutropenia, thrombocytopenia and associated deaths from hemorrhage remains an unresolved clinical problem given limited therapeutic options. There are currently no applicable cytokines approved by FDA in enhancing thrombopoiesis except for interleukin 11, which has not been utilized clinically due to its serious adverse effects (Schwertschlag et al., “Interleukin 11” In Platelets, Michelson, ed., San Diego, Academic Press, pp. 845-854 (2002). Frequent platelet transfusions are the only option, but the shelf life of fresh platelets is only 5 days in refrigeration, and only 2 days after screening for transmittable pathogens. In a mass nuclear event, the demand for fresh platelets will overwhelm the nation's supply of fresh platelets. Developing mitigating agents to accelerate the recovery of progenitor and precursor cells for thrombopoiesis will be vital as a countermeasure of Acute Radiation Syndrome (ARS).

Thrombopoietin (TPO) is the key endogenous thrombopoietic cytokine and a ligand that binds to and activates the proto-oncogene cytokine receptor c-Mpl (de Sauvage et al., “Stimulation of Megakaryocytopoiesis and Thrombopoiesis by the c-Mpl Ligand,” Nature 369(6481):533-538 (1994); Kaushansky et al., “Promotion of megakaryocyte progenitor Expansion and Differentiation by the c-Mpl Ligand Thrombopoietin,” Nature 369(6481):568-571 (1994); Sohma et al., “Molecular Cloning and Chromosomal Localization of the Human Thrombopoietin Gene,” FEBS Letters 353(1):57-61 (1994). The c-Mpl receptor genes have been cloned for both mouse and human. The receptor contains the extracellular domain, the transmembrane (TM) domain and the cytoplasmic intracellular domain (Mignotte et al., “Structure and Transcription of the Human c-mpl Gene (MPL),” Genomics 20:5-12 (1994); Vigon et al., “Characterization of the Murine Mpl Proto-oncogene, a Member of the Hematopoietic Cytokine Receptor Family: Molecular Cloning, Chromosomal Location and Evidence for a Function in Cell Growth,” Oncogene 8:2607-15 (1993); Li et al., “Cloning and Functional Characterization of a Novel c-mpl Variant Expressed in Human CD34 Cells and Platelets,” Cytokine 12(7):835-44 (2000); Alexander & Dunn, “Structure and Transcription of the Genomic Locus Encoding Murine c-Mpl, a Receptor for Thrombopoietin. Oncogene 10:795-803 (1995)). Recombinant human thrombopoietin (rhTPO) and its shorter, pegylated recombinant megakaryocyte growth and development factor (PEG-rhMGDF) were developed, but unfortunately were associated with autoantibody formation (Basser et al., “Development of Pancytopenia with Neutralizing Antibodies to Thrombopoietin After Multicycle Chemotherapy Supported by Megakaryocyte Growth and Development Factor,” Blood 99(7):2599-2602 (2002)). For this reason, clinical trials of these agents have been discontinued in the United States.

Stimulating platelet production remains an unmet clinical need in the management of thrombocytopenia. Second generation thrombopoietic growth factors with unique pharmacological properties have been developed, which include peptide mimetics, such as AMG531 (Cohn & Bussel, “Romiplostim: A Second-generation Thrombopoietin Agonist,” Drugs Today (Barc), 45(3):175-88 (2009)), which activates the cMpl (TPO receptor) through the extracellular domain, and the TPO nonpeptide mimetics, such as NIP-004, eltrombopag and other small molecules (Yamane et al., “Characterization of Novel Non-peptide Thrombopoietin Mimetics, Their Species Specificity and the Activation Mechanism of the Thrombopoietin Receptor,” Eur J Pharmacol 586(1-3):44-51 (2008); Erickson-Miller et al., “Discovery and Characterization of a Selective, Nonpeptidyl Thrombopoietin Receptor Agonist,” Exp Hematol 33(1):85-93 (2005)). These non-peptide TPO mimetics bind and activate the cMpl trans-membrane (TM) domain instead of the extracellular domain (Mignotte et al., “Structure and Transcription of the Human c-mpl Gene (MPL),” Genomics 20:5-12 (1994); Vigon et al., “Characterization of the Murine Mpl Proto-oncogene, a Member of the Hematopoietic Cytokine Receptor Family: Molecular Cloning, Chromosomal Location and Evidence for a Function in Cell Growth,” Oncogene 8:2607-15 (1993); Li et al., “Cloning and Functional Characterization of a Novel c-mpl Variant Expressed in Human CD34 Cells and Platelets,” Cytokine 12(7):835-44 (2000); Alexander & Dunn, “Structure and Transcription of the Genomic Locus Encoding Murine c-Mpl, a Receptor for Thrombopoietin. Oncogene 10:795-803 (1995)). These newer agents increase platelet counts by binding and activating the TPO receptor (TPO-R), c-Mpl. However, none of the newer thrombopoietic agents have been reported to enhance post-radiation thrombopoiesis. This is due, in part, to the species specificity of these newer agents that limits the experimental animal models for radiation investigations.

While the development of a mitigating agent that is effective and is ideal for national stockpile for Acute Radiation Syndrome (ARS) indication is highly desirable, the lack of suitable animal models (except for chimpanzee, which is a protected species) for radiation investigation presents a major challenge. Animal models that overcome the species specificity are critical to the product development of all TPO mimetics, but particularly for the development of agents that can be used to treat ARS.

The present invention is directed at overcoming these and other deficiencies in the art.

### SUMMARY OF THE INVENTION

A first aspect of the present invention relates to a transgenic non-human mammal. The transgenic non-human mammals of the invention are particularly useful for the screening of thrombopoietin mimetics, thrombopoietin receptor agonists, or thrombopoietin receptor antagonists active on the human thrombopoietin receptor.

According to one embodiment, the transgenic non-human mammal has a genome that includes a stably integrated transgene construct including a polynucleotide sequence encoding a humanized thrombopoietin receptor wherein the transgenic non-human mammal has a baseline blood platelet count corresponding to a physiological blood platelet count of a matched non-transgenic non-human mammal.

According to another embodiment, the transgenic non-human mammal has a genome that includes a stably integrated transgene construct including a polynucleotide sequence encoding a chimeric thrombopoietin receptor, wherein the chimeric thrombopoietin receptor includes extracellular and transmembrane domains of a human thrombopoietin receptor operably coupled to a cytoplasmic domain of a non-human thrombopoietin receptor.

A second aspect of the present invention relates to an isolated cell or tissue derived from the transgenic non-human mammal according to the first aspect of the invention. Also encompassed by this aspect of the invention are transgenic host cells that contain the transgene construct having a polynucleotide sequence encoding a humanized thrombopoietin receptor.

A third aspect of the present invention relates to a method of identifying a human thrombopoietin mimetic, thrombopoietin receptor agonist, or a thrombopoietin receptor antagonist. This method includes administering a candidate compound to isolated cells or tissue derived from the transgenic non-human mammal according to the first aspect of the invention; measuring one or more endpoints selected from the group consisting of cell proliferation level, cell differentiation level, and gene expression level in the isolated cells or tissue after said administering; comparing the measured one or more end-points to one or more corresponding end-points in a reference sample; and identifying a human thrombopoietin mimetic, thrombopoietin receptor agonist, or a thrombopoietin receptor antagonist based on said comparing.

A fourth aspect of the present invention relates to a method of identifying a human thrombopoietin mimetic, thrombopoietin receptor agonist, or a thrombopoietin receptor antagonist. This method includes administering a candidate compound to the transgenic non-human mammal according to the first aspect of the invention; obtaining a cell count in the transgenic non-human mammal after said administering; comparing the obtained cell count to a reference cell count; and identifying a human thrombopoietin mimetic, thrombopoietin receptor agonist, or a thrombopoietin receptor antagonist based on said comparing.

A fifth aspect of the present invention relates to a method of identifying a human thrombopoietin mimetic, thrombopoietin receptor agonist, or a thrombopoietin receptor antagonist. This method includes administering a candidate compound to the transgenic non-human mammal according to the first aspect of the invention; measuring one or more endpoints selected from the group consisting of cell proliferation, cell differentiation, and gene expression in one or more cell types or tissues of the transgenic non-human mammal after said administering; comparing the one or more measured endpoints to one or more corresponding endpoints in one or more cell types or tissues of a control non-human mammal; and identifying a human thrombopoietin mimetic, thrombopoietin receptor agonist, or a thrombopoietin receptor antagonist based on said comparing.

A sixth aspect of the present invention relates to a method of identifying a human thrombopoietin mimetic, thrombopoietin receptor agonist, or a thrombopoietin receptor antagonist. This method includes administering a candidate compound to the transgenic non-human mammal according to the first aspect of the invention; measuring one or more endpoints selected from the group consisting of cell repair, tissue repair and/or regeneration, and organ repair and/or regeneration in one or more cell types, tissues, or organs of the transgenic non-human mammal after said administering; comparing the one or more measured endpoints to one or more corresponding endpoints in one or more cell types, tissues, or organs of a control non-human mammal; and identifying a human thrombopoietin mimetic, thrombopoietin receptor agonist, or a thrombopoietin receptor antagonist based on said comparing.

A seventh aspect of the present invention relates to a method of treating a subject for acute radiation syndrome that includes administering a c-Mpl receptor agonist to the subject under conditions effective to treat acute radiation syndrome. This aspect may also include administering cell therapy, cytokine(s) or immune modulator(s) prior to, concurrently with, or after said administering the c-Mpl receptor agonist.

An eighth aspect of the present invention relates to a method of treating a subject for chronic radiation syndrome that includes administering a c-Mpl receptor agonist to the subject under conditions effective to treat chronic radiation syndrome. This aspect may also include administering cell therapy, cytokine(s) or immune modulator(s) prior to, concurrently with, or after said administering the c-Mpl receptor agonist.

A ninth aspect of the present invention relates to a method of treating a subject having a bone marrow injury resulting from exposure to a non-therapeutic chemical agent. This method includes administering a c-Mpl receptor agonist to the subject under conditions effective to treat the bone marrow injury resulting from exposure to the non-therapeutic chemical agent. This aspect may also include administering a aspect may also include administering cell therapy, cytokine(s), or immune modulator(s) prior to, concurrently with, or after said administering the c-Mpl receptor agonist.

A tenth aspect of the present invention relates to a method of inducing tissue repair or tissue regeneration in a subject that includes administering a c-Mpl receptor agonist to the subject under conditions effective to induce tissue repair or tissue regeneration in the subject. This aspect may also include administering aspect may also include administering cell therapy, cytokine(s), or immune modulator(s) prior to, concurrently with, or after said administering the c-Mpl receptor agonist.

As demonstrated in the accompanying Examples, the development of a human TPO-R, c-Mpl, TM knock in (KI) mouse model (Mpl^{hExon10}) represents a significant advance for the screening human TPO mimetics. This mouse model, unlike other TPO receptor mouse models, exhibits a baseline blood platelet count corresponding to a physiological blood platelet count (e.g., about 300×10³/μl to about 1600×10³/μl) of a corresponding, matched non-transgenic mouse. This allows for a direct comparison of the effect of TPO mimetics in both the transgenic mouse model and control mouse, which prior to the present invention has not been possible.

The development of a human TPO-R, c-Mpl, knock in (KI) mouse model (Mpl^{hmMPL}) represents a significant advance for the screening human TPO mimetics. This mouse model, unlike other TPO receptor mouse models, exhibits supraphysiological blood platelet count (e.g., above 1600×10³/μl) of a corresponding, matched non-transgenic mouse. This model is useful for screening TPO peptide mimetics or antibodies to TPO receptor.

### BRIEF DESCRIPTION OF THE DRAWINGS

FIG. 1 depicts the generation of the Mpl^{hmMPL} knock-in allele used to produce the human thrombopoietin receptor knock-in mouse. Wildtype mouse Mpl genomic structure and restriction enzyme map are shown at the top. The open box represents the 5′ untranslated region (5′ UTR), and the filled boxes are the coding regions, with each exon numerically labeled. The hatched box shows the 3′ Southern blot probe. The thick white and black bars are the sequences used to generate the 5′ and 3′ homologous arms in the targeting construct, respectively. The Mpl^{hmMIPL} targeting vector is made by inserting human/mouse Mpl hybrid cDNA (red box “hmMPL”)/SV40 polyadenylation fusion sequences and the foxed pGK-Neo sequences at XhoI and SalI sites in the pKII targeting vector. CMV-Cre mice are used to remove the pGK-Neo in Mpl^{hmMPLNeo} mice, to generate the Mpl^{hmMPL} knock-in allele. Abbreviations: Neo, pGK-neomycin resistance gene; DTA, pGK-diphtheriatoxin gene for negative selection of ES cells; loxP (triangles), Cre recombinase recognition sequences; and pA,SV40 polyadenylation sequences

FIGS. 2A-2B show experimental verification of human TPO receptor cDNA knock-in mouse Mpl^{hmMPL} at the DNA level. FIG. 2A is an alignment of genomic DNA PCR primer sequences (italicized) with the 5′-ends of mouse genome (SEQ ID NO: 8) and human cDNA (SEQ ID NO: 9) sequences of c-mpl (exon 1, introns 1-2, and exon 2). The forward primer (SEQ ID NO: 10) is located at the 5′-end untranscribed sequence upstream of the mouse c-mpl gene. The reverse primer (SEQ ID NO: 11) corresponds to an exon 2 sequence where human and mouse genes are identical. The reverse primer sequence is complementary to the sense strands. The PCR products are 309 bp for human cDNA KI mice and 462 bp for wild-type mice, respectively. The start codon ATG is underlined. The exon sequences are bolded. FIG. 2B is shows an agarose gel electrophoresis analysis of the PCR products.

FIG. 3 is a Kaplan-Meier survival curve showing survival of wildtype and Mpl^{hmMPL} knock-in mice after exposure to a non-therapeutic dose of radiation. Male Mpl^{hmMPL} knock-in mice received total body irradiation (TBI) of 8 Gy. Twenty-four hours after TBI, mice were gavaged daily with eltrombopag (50 mg/kg) for 15 days. Survival of mice was monitored twice daily for 30 days. Experiments were carried out with 8 homozygous Mpl^{hmMPL} knock-in mice, 10 heterozygous Mpl^{hmMPL} knock-in mice, and 10 wild type mice.

FIG. 4 shows an alignment of human and mouse c-mpl exon 10 DNA (SEQ ID NOs: 12 and 13, respectively) and the encoded amino acid sequences (SEQ ID NOs: 14 and 15, respectively). The gene segment encoding trans-membrane (TM) domain in each nucleotide sequence is underlined. Stars under the aligned sequences indicate identical nucleotide bases or amino acid residues. Differences between human and mouse sequences are shaded.

FIG. 5 shows the human exon 10 sense (SEQ ID NO: 16) and antisense (SEQ ID NO: 17) oligonucleotides with flanking sequences corresponding to mouse introns 9-10 and 10-11, respectively. These oligonucleotides were synthesized, annealed and subcloned as a 169 bp fragment into EcoRI and BamHI sites (italicized) of pBluescript SK vector. Human exon10 sequence with flanking mouse intron sequences are indicated by arrows and the segment encoding the transmembrane domain is underlined. The SmaI and KpnI (bold) fragment containing the synthetic human exon10 and the flanking mouse intron sequences were used to replace the mouse SmaI-Exon10-KpnI sequences.

FIG. 6 depicts the generation of the Mpl^{hExon10} knock-in allele. The mouse c-mpl genomic structure and restriction enzyme map covering c-mpl exons 7-12 is shown at the top. c-mpl exons are shown as boxes. Open box represents the 3′ UTR and the filled boxes are the coding regions. Human exon 10 is shown as a filled box in red. The hatched box shows the 5′ Southern probe. The thick white and black bars are the sequences used to generate the 5′ and 3′ homologous arms in the targeting construct, respectively. The Mpl^{hExon10} targeting vector is made by replacing mouse c-mpl exon 10 with human c-mpl exon 10 (red box) and inserting a floxed pGK-Neo sequence in intron 10. CMV-Cre mice are used to remove the pGK-Neo in Mpl^{hExon10Neo} mice (human TM knockin mouse) to generate Mpl^{hExon10} knock-in allele. Abbreviations: Neo, pGK neomycin resistance gene; DTA, pGK-diphtheria toxin gene for negative selection of ES cells; loxP (triangles), and Cre recombinase recognition sequences.

FIG. 7 is an agarose gel electrophoresis analysis of the PCR products generated from wildtype, heterozygous, and homozygous human TPO receptor (c-Mpl) exon 10 knockin mice genomic DNA (Mpl^{hExon10}).

FIG. 8 is an agarose gel electrophoresis analysis of the RT-PCR products. All bone marrow cDNA samples from wild-type, heterozygote, and homozygote c-mpl exon 10 knock-in mice (Mpl^{hExon10}) tested positive for β-actin, a house keeping gene used as a positive control (lanes 2, 5, and 8, respectively).

FIGS. 9A-9C show gel electrophoresis analysis of the RT-PCR products (FIG. 9A) and alignment of c-mpl exon 10 cDNA sequences and its flanking regions (FIGS. 9B and 9C). The sequence alignment of FIG. 9B (sense strand) aligns (1) human c-mpl exon 10 sequence, SEQ ID NO: 12; (2) experimentally determined sequence of exon 10 cDNA and its flanking region of the KI mouse, SEQ ID NO: 18; (3) experimentally determined sequence of exon 10 cDNA and its flanking region of wild-type mouse, SEQ ID NO: 19; and (4) mouse sequence of exon 10 and its flanking regions, SEQ ID NO: 20. The sequence alignment of FIG. 9C (antisense strands) aligns (1) human c-mpl exon 10 sequence, SEQ ID NO: 21; (2) experimentally determined sequence of exon 10 cDNA and its flanking region of the KI mouse, SEQ ID NO: 22; (3) experimentally determined sequence of exon 10 cDNA and its flanking region of wild-type mouse, SEQ ID NO: 23; and (4) mouse sequence of exon 10 and its flanking regions, SEQ ID NO: 24. The gene fragment encoding the trans-membrane domain is underlined. The nucleotide mismatches between the mouse and human exon 10 sequences are shaded. The primer sequences used for both RT-PCR and DNA sequencing are bolded. The RT-PCR product is 258 bp. Stars below the sequences indicate identical nucleotides.

FIGS. 10A-10D show that eltrombopag increases baseline platelet, bone marrow, CD41+34− cells and stem cells in the human c-mpl exon 10 knock-in mouse (Mpl^{hExon10}). FIG. 10A is a graph showing that eltrombopag significantly increased the platelet counts in the peripheral blood of the homozygous human TPO receptor (c-mpl) exon 10 knock-in mice Mpl^{hExon10} (c-Mpl TM KI mutant). It is noteworthy that the platelet count in mutant is not significantly different from that of the wild-type. FIG. 10B is a graph showing showed that eltrombopag significantly increased the bone marrow CD41⁺CD42⁺ cells in the homozygous Mpl^{hExon10}KI mice (c-Mpl TM KI mutant). FIGS. 10C-D show that eltrombopag significantly increased the bone marrow Lin⁻KSL (stem) cells in the Mpl^{hExon10}KI (c-Mpl TM KI mutant).

FIG. 11 shows eltrombopag improves survival of irradiated Mpl^{hExon10}KI (human c-mpl TM-knock-in mice). Male Mpl^{hExon10}KI (human c-mpl TM knock-in mice) received 7.75 Gy TBI. Twenty-four hours after irradiation, mice were gavaged with either vehicle (0 mg/kg of eltrombopag) or one of the three doses of eltrombopag (12.5 mg/kg, 25 m g/kg, vs. 50 mg/kg daily for 15 days (n=8-10) for each experimental group.

FIGS. 12A-D illustrate the effects of IL-11 only (middle/red bars) versus TPO+IL11 (left/blue bars) versus eltrombopag (8 μg/mL)+IL11 (right/green bars) on promoting megakaryocyte or CD41+CD34− cell differentiation in control and irradiated ex vivo human 3D bone marrow mononuclear cells. Cells were treated with TPO and IL11 (5 ng/mL each) for 6-7 days to induce megakaryocyte differentiation. Culture media was replaced with cultures containing IL-11 only, TPO+IL11, or eltrombopag+IL11 for the groups of control cultures (FIGS. 12A, 12C) and the group of irradiated cultures (FIGS. 12B, 12D). The cultures were maintained for another 14 days and screened for the presence of megakaryocytes and CD41+CD34− cells (precursors/progenitors for thrombopoiesis) every 7 days. Each culture was set up in three replicates.

FIG. 13 is a graph showing weekly platelet counts for human TM KI mice after 6.5 Gy of total body irradiation (TBI) and treatment with either eltrombopag or distilled water (vehicle; left/blue bars) 24 hours after TBI. The data are presented as mean±standard error of the mean. * P=0.05, ** P=0.02 two-tail paired t-test.

FIG. 14 is a graph showing weekly red blood cell counts (RBC) for human TM KI mice after 6.5 Gy of total body irradiation (TBI) and treatment with either eltrombopag or distilled water (vehicle; left/blue bars) 24 hours after TBI. The data are presented as mean±standard error of the mean. * P=0.04, two-tail paired t-test.

FIG. 15 is a graph showing weekly white blood cell counts (WBC) for human TM KI mice after 6.5 Gy of total body irradiation (TBI) and treatment with either eltrombopag or distilled water (vehicle; left/blue bars) 24 hours after TBI. The data are presented as mean±standard error of the mean. ** P<0.01, two-tail paired t-test.

FIG. 16 is a graph showing weekly counts of bone marrow CD41+CD42+ cells of human TM KI mice after 6.5 Gy of total body irradiation (TBI) and treatment with either eltrombopag or distilled water (vehicle; left/blue bars) 24 hours after TBI. The data are presented as mean±standard error of the mean. * P=0.03, two-tail paired t-test.

### DETAILED DESCRIPTION OF THE INVENTION

A first aspect of the present invention relates to a transgenic non-human mammal whose genome includes a stably integrated expression construct having a polynucleotide sequence encoding a humanized thrombopoietin (“TPO”) receptor wherein the transgenic non-human mammal has a baseline blood platelet count corresponding to a physiological blood platelet count of a matched non-transgenic non-human mammal. As used herein, the term “matched” means that the non-transgenic mammal is the same background strain as used to generate the transgenic animal or a closely related strain that is, with respect to platelet counts, indistinguishable.

The transgenic non-human mammal of the present invention can be any non-human mammal including, but not limited to, a mouse, rat, rabbit, guinea pig, pig, micro-pig, goat, and non-human primate, e.g., a baboon, monkey, and chimpanzee. In one embodiment of the present invention, the non-human mammal is a rodent, preferably a rat or mouse. Suitable strains of mice commonly used in the generation of transgenic models include, without limitation, CD-1® Nude mice, NU/NU mice, BALB/C Nude mice, BALB/C mice, NIH-III mice, SCID® mice, outbred SCID® mice, SCID Beige mice, C3H mice, C57BL/6 mice, DBA/2 mice, FVB mice, CB17 mice, 129 mice, SJL mice, B6C3F1 mice, BDF1 mice, CDF1 mice, CB6F1 mice, CF-1 mice, Swiss Webster mice, SKH1 mice, PGP mice, and B6SJL mice.

In one embodiment of the present invention, the humanized TPO receptor of the transgenic animal includes at least a portion of human TPO receptor exon 10. In accordance with this embodiment of the present invention, the at least a portion of the human TPO receptor exon 10 includes one or more consecutive or non-consecutive amino acid residues of the human thrombopoietin receptor exon 10. Exon 10 of the human TPO receptor is shown in bold within the full-length amino acid sequence of the human TPO receptor of SEQ ID NO:1 (below). In one embodiment of the present invention, the at least a portion of the human TPO receptor exon 10 includes an amino acid residue corresponding to the histidine residue at position 499 of SEQ ID NO:1.

SEQ ID NO: 1
Human Thrombopoietin Receptor

| | |
|---|---|
| MPSWALFMVT SCLLLAPQNL AQVSSQDVSL LASDSEPLKC | 50 |
| FSRTFEDLTC | |
| FWDEEEAAPS GTYQLLYAYP REKPRACPLS SQSMPHFGTR | 100 |
| YVCQFPDQEE | |
| VRLFFPLHLW VKNVFLNQTR TQRVLFVDSV GLPAPPSIIK | 150 |
| AMGGSQPGEL | |
| QISWEEPAPE ISDFLRYELR YGPRDPKNST GPTVIQLIAT | 200 |
| ETCCPALQRP | |
| HSASALDQSP CAQPTMPWQD GPKQTSPSRE ASALTAEGGS | 250 |
| CLISGLQPGN | |
| SYWLQLRSEP DGISLGGSWG SWSLPVTVDL PGDAVALGLQ | 300 |
| CFTLDLKNVT | |
| CQWQQQDHAS SQGFFYHSRA RCCPRDRYPI WENCEEEEKT | 350 |
| NPGLQTPQFS | |
| RCHFKSRNDS IIHILVEVTT APGTVHSYLG SPFWIHQAVR | 400 |
| LPTPNLHWRE | |
| ISSGHLELEW QHPSSWAAQE TCYQLRYTGE GHQDWKVLEP | 450 |
| PLGARGGTLE | |
| LRPRSRYRLQ LRARLNGPTY QGPWSSWSDP TRVETATETA | 500 |
| WISLVTALHL | |
| VLGLSAVLGL LLLRWQFPAH YRRLRHALWP SLPDLHRVLG | 550 |
| QYLRDTAALS | |
| PPKATVSDTC EEVEPSLLEI LPKSSERTPL PLCSSQAQMD | 600 |
| YRRLQPSCLG | |
| TMPLSVCPPM AESGSCCTTH IANHSYLPLS YWQQP | 635 |

In another embodiment of the present invention, the humanized TPO receptor of the transgenic animal includes at least a portion of the human TPO receptor transmembrane domain. In accordance with this embodiment of the present invention, the at least a portion of the human TPO receptor transmembrane domain includes one or more consecutive or non-consecutive amino acid residues of the human TPO transmembrane domain. The transmembrane domain of the human TPO receptor is underlined in SEQ ID NO: 1 above. In one embodiment of the present invention, the transgenic non-human animal includes a humanized thrombopoietin receptor where the transmembrane domain of the humanized receptor has an amino acid sequence of SEQ ID NO:2.

SEQ ID NO: 2
Transmembrane Domain of Humanized
Thrombopoietin Receptor
I [S/T] L V T A L L H L V L [G/S] L S A
[V/L] L G L L L L

In one embodiment of the present invention, the humanized TPO receptor comprises a humanized-mouse TPO receptor. The amino acid sequence of the mouse TPO receptor is provided below as SEQ ID NO: 3. In accordance with this embodiment of the present invention, all or a portion of exon 10 of the mouse TPO receptor (shown in bold) or the transmembrane domain of the receptor (underlined) is replaced with one or more consecutive or non-consecutive amino acid residues of the human TPO receptor exon 10 or transmembrane domain shown above.

SEQ ID NO: 3
Mouse Thrombopoietin Receptor

| | |
|---|---|
| MPSWALFMVT SCLLLALPNQ AQVTSQDVFL LALGTEPLNC | 50 |
| FSQTFEDLTC | |
| FWDEEEAAPS GTYQLLYAYR GEKPRACPLY SQSVPTFGTR | 100 |
| YVCQFPAQDE | |
| VRLFFPLHLW VKNVSLNQTL IQRVLFVDSV GLPAPPRVIK | 150 |
| ARGGSQPGEL | |
| QIHWEAPAPE ISDFLRHELR YGPTDSSNAT APSVIQLLST | 200 |
| ETCCPTLWMP | |
| NPVPVLDQPP CVHPTASQPH GPVRTSPAGE APFLTVKGGS | 250 |
| CLVSGLQAGK | |
| SYWLQLRSQP DGVSLRGSWG PWSFPVTVDL PGDAVTIGLQ | 300 |
| CFTLDLKMVT | |
| CQWQQQDRTS SQGFFRHSRT RCCPTDRDPT WEKCEEEEPR | 350 |
| PGSQPALVSR | |
| CHFKSRNDSV IHILVEVTTA QGAVHSYLGS PFWIHQAVLL | 400 |
| PTPSLHWREV | |
| SSGRLELEWQ HQSSWAAQET CYQLRYTGEG REDWKVLEPS | 450 |
| LGARGGTLEL | |
| RPRARYSLQL RARLNGPTYQ GPWSAWSPPA RVSTGSETAW | 500 |
| ITLVTALLLV | |
| LSLSALLGLL LLKWQFPAHY RRLRHALWPS LPDLHRVLGQ | 550 |
| YLRDTAALSP | |
| SKATVTDSCE EVEPSLLEIL PKSSESTPLP LCPSQPQMDY | 600 |
| RGLQPCLRTM | |
| PLSVCPPMAE TGSCCTTHIA NHSYLPLSYW QQP | 633 |

In one embodiment of the present invention, the humanized mouse TPO receptor includes an amino acid sequence of SEQ ID NO: 4: as follows:

SEQ ID NO: 4 AA Sequence of the Chimeric TPO Receptor in
the hMPL^{hTM} Mouse

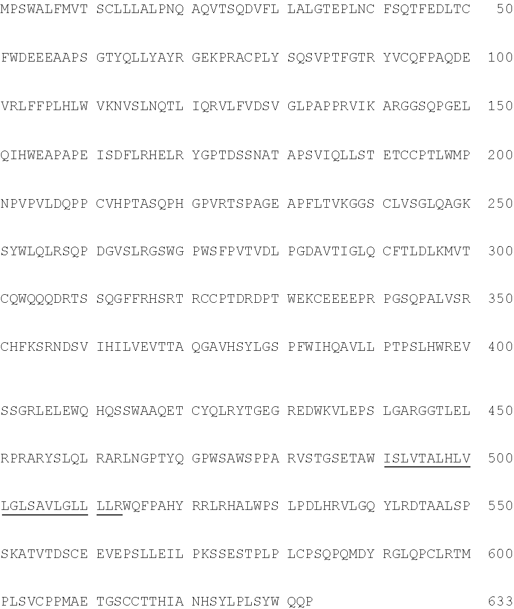

The transmembrane domain is underlined. The five human-specific amino acid residues in exon 10 are shaded.

A polynucleotide sequence encoding the open reading frame of the humanized mouse TPO receptor of SEQ ID NO: 4 includes a nucleotide sequence of SEQ ID NO: 5 as follows:

| | |
|---|---|
| ATGCCCTCTT GGGCCCTCTT CATGGTCACC TCCTGCCTCC TCTTGGCCCT | 50 |
| TCCAAACCAG GCACAAGTCA CCAGCCAAGA TGTCTTCTTG CTGGCCTTGG | 100 |
| GCACAGAGCC CCTGAACTGC TTCTCCCAAA CATTTGAGGA CCTCACCTGC | 150 |
| TTCTGGGATG AGGAAGAGGC AGCACCCAGT GGGACATACC AGCTGCTGTA | 200 |
| TGCCTACCGA GGAGAGAAGC CCCGTGCATG CCCCCTGTAT TCCCAGAGTG | 250 |
| TGCCCACCTT TGGAACCCGG TATGTGTGCC AGTTTCCAGC CCAGGATGAA | 300 |
| GTGCGCCTCT TCTTTCCGCT GCACCTCTGG GTGAAGAATG TGTCCCTCAA | 350 |
| CCAGACTTTG ATCCAGCGGG TGCTGTTTGT GGATAGTGTG GGCCTGCCAG | 400 |
| CTCCCCCCAG GGTCATCAAG GCCAGGGGTG GGAGCCAACC AGGGGAACTT | 450 |
| CAGATCCACT GGGAGGCCCC TGCTCCTGAA ATCAGTGACT TCCTGAGGCA | 500 |
| TGAACTCCGC TATGGCCCCA CGGACTCCAG CAACGCCACT GCCCCCTCCG | 550 |
| TCATTCAGCT GCTCTCCACA GAAACCTGCT GCCCCACTTT GTGGATGCCG | 600 |
| AACCCAGTCC CTGTTCTTGA CCAGCCTCCG TGTGTTCATC CGACAGCATC | 650 |
| CCAACCGCAT GGACCAGTGA GGACCTCCCC AGCTGGAGAA GCTCCATTTC | 700 |
| TGACAGTGAA GGGTGGAAGC TGTCTCGTCT CAGGCCTCCA GGCTGGCAAA | 750 |
| TCCTACTGGC TCCAGCTACG CAGCCAACCC GACGGGGTCT CCCTTCGTGG | 800 |
| CTCCTGGGGA CCCTGGTCCT TCCCTGTGAC TGTGGATCTT CCAGGAGATG | 850 |
| CAGTGACAAT TGGACTTCAG TGCTTTACCT TGGATCTGAA GATGGTCACC | 900 |
| TGCCAGTGGC AGCAACAAGA CCGCACTAGC TCCCAAGGCT TCTTCCGCCA | 950 |
| CAGCAGGACG AGGTGCTGCC CCACAGACAG GGACCCCACC TGGGAGAAAT | 1000 |
| GTGAAGAGGA GGAACCGCGT CCAGGATCAC AGCCCGCTCT CGTCTCCCGC | 1050 |
| TGCCACTTCA AGTCACGAAA TGACAGTGTT ATTCACATCC TTGTAGAGGT | 1100 |
| GACCACAGCG CAAGGTGCCG TTCACAGCTA CCTGGGCTCC CCTTTTTGGA | 1150 |
| TCCACCAGGC TGTGCTCCTT CCCACCCCGA GCCTGCACTG GAGGGAGGTC | 1200 |
| TCAAGTGGAA GGCTGGAGTT GGAGTGGCAG CACCAGTCAT CTTGGGCAGC | 1250 |
| TCAAGAGACC TGCTACCAGC TCCGGTACAC GGGAGAAGGC CGTGAGGACT | 1300 |
| GGAAGGTGCT GGAGCCATCT CTCGGTGCCC GGGGAGGGAC CCTAGAGCTG | 1350 |
| CGCCCCCGAG CTCGCTACAG CTTGCAGCTG CGTGCCAGGC TCAACGGCCC | 1400 |
| CACCTACCAA GGTCCCTGGA GCGCCTGGTC TCCCCCAGCT AGGGTGTCCA | 1450 |
| 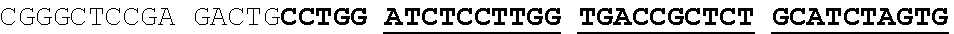 | 1500 |
| 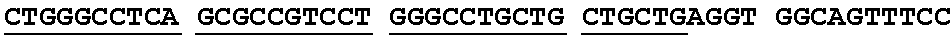 | 1550 |
| 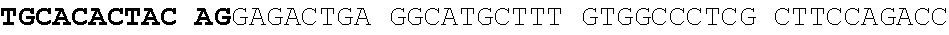 | 1600 |
| TACACCGGGT CCTAGGCCAG TACCTCAGAG ACACTGCAGC CCTAAGTCCT | 1650 |
| TCTAAGGCCA CGGTTACCGA TAGCTGTGAA GAAGTGGAAC CCAGCCTCCT | 1700 |
| GGAAATCCTC CCTAAGTCCT CAGAGAGCAC TCCTTTACCT CTGTGTCCCT | 1750 |
| CCCAACCTCA GATGGACTAC AGAGGACTGC AACCTTGCCT GCGGACCATG | 1800 |
| CCCCTGTCTG TGTGTCCACC CATGGCTGAG ACGGGGTCCT GCTGCACCAC | 1850 |
| ACACATTGCC AACCACTCCT ACCTACCACT AAGCTATTGG CAGCAGCCCT | 1900 |
| GA | 1902 |

The sequence encoding the transmembrane domain is underlined, and the sequence of exon 10 appears in bold typeface. The nucleotide base changes that encode for the five human-specific amino acid residues in exon 10 are shaded.

Another aspect of the present invention relates to a transgenic non-human mammal whose genome includes a stably integrated expression construct including a polynucleotide sequence encoding a chimeric thrombopoietin receptor, wherein the chimeric thrombopoietin receptor includes extracellular and transmembrane domains of a human thrombopoietin receptor operably coupled to a cytoplasmic domain of a non-human thrombopoietin receptor.

In one embodiment of this aspect of the present invention, the transgenic non-human mammal includes a chimeric mouse-human thrombopoietin receptor. For example, as described herein, a suitable chimeric mouse-human TPO receptor includes extracellular and transmembrane domains of the human TPO receptor coupled to the cytoplasmic domain of the mouse TPO receptor. The amino acid sequence encoding this mouse-human chimeric TPO receptor is shown below as SEQ ID NO: 6 as follows:

SEQ ID NO: 6
AA Sequence of the Chimeric TPO
Receptor in the hMPL^{cDNA} Mouse

| | |
|---|---|
| MPSWALFMVT SCLLLAPQNL AQVSSQDVSL LASDSEPLKC | 50 |
| FSRTFEDLTC | |
| FWDEEEAAPS GTYQLLYAYP REKPRACPLS SQSMPHFGTR | 100 |
| YVCQFPDQEE | |
| VRLFFPLHLW VKNVFLNQTR TQRVLFVDSV GLPAPPSIIK | 150 |
| AMGGSQPGEL | |
| QISWEEPAPE ISDFLRYELR YGPRDPKNST GPTVIQLIAT | 200 |
| ETCCPALQRP | |
| HSASALDQSP CAQPTMPWQD GPKQTSPSRE ASALTAEGGS | 250 |
| CLISGLQPGN | |
| SYWLQLRSEP DGISLGGSWG SWSLPVTVDL PGDAVALGLQ | 300 |
| CFTLDLKNVT | |
| CQWQQQDHAS SQGFFYHSRA RCCPRDRYPI WENCEEEEKT | 350 |
| NPGLQTPQFS | |
| RCHFKSRNDS IIHILVEVTT APGTVHSYLG SPFWIHQAVR | 400 |
| LPTPNLHWRE | |
| ISSGHLELEW QHPSSWAAQE TCYQLRYTGE GHQDWKVLEP | 450 |
| PLGARGGTLE | |
| LRPRSRYRLQ LRARLNGPTY QGPWSSWSDP TRVETATETA | 500 |
| WISLVTALHL | |
| VLGLSAVLGL LLLKWQFPAH YRRLRHALWP SLPDLHRVLG | 550 |
| QYLRDTAALS | |
| PSKATVTDSC EEVEPSLLEI LPKSSESTPL PLCPSQPQMD | 599 |
| YRGLQP-CLR | |
| TMPLSVCPPM AETGSCCTTH IANHSYLPLS YWQQP | 634 |

The human extracellular and transmembrane domain sequences are bolded, and the transmembrane domain is also underlined.

In accordance with this aspect of the present invention, a polynucleotide sequence encoding the open reading frame of the chimeric thrombopoietin receptor of SEQ ID NO: 6 includes a nucleotide sequence of SEQ ID NO: 7 as follows:

| | |
|---|---|
| ATGCCCTCCT GGGCCCTCTT CATGGTCACC TCCTGCCTCC TCCTGGCCCC | 50 |
| TCAAAACCTG GCCCAAGTCA GCAGCCAAGA TGTCTCCTTG CTGGCATCAG | 100 |
| ACTCAGAGCC CCTGAAGTGT TTCTCCCGAA CATTTGAGGA CCTCACTTGC | 150 |
| TTCTGGGATG AGGAAGAGGC AGCGCCCAGT GGGACATACC AGCTGCTGTA | 200 |
| TGCCTACCCG CGGGAGAAGC CCCGTGCTTG CCCCCTGAGT TCCCAGAGCA | 250 |
| TGCCCCACTT TGGAACCCGA TACGTGTGCC AGTTTCCAGA CCAGGAGGAA | 300 |
| GTGCGTCTCT TCTTTCCGCT GCACCTCTGG GTGAAGAATG TGTTCCTAAA | 350 |
| CCAGACTCGG ACTCAGCGAG TCCTCTTTGT GGACAGTGTA GGCCTGCCGG | 400 |
| CTCCCCCCAG TATCATCAAG GCCATGGGTG GGAGCCAGCC AGGGGAACTT | 450 |
| CAGATCAGCT GGGAGGAGCC AGCTCCAGAA ATCAGTGATT TCCTGAGGTA | 500 |
| CGAACTCCGC TATGGCCCCA GAGATCCCAA GAACTCCACT GGTCCCACGG | 550 |
| TCATACAGCT GATTGCCACA GAAACCTGCT GCCCTGCTCT GCAGAGGCCT | 600 |
| CACTCAGCCT CTGCTCTGGA CCAGTCTCCA TGTGCTCAGC CCACAATGCC | 650 |
| CTGGCAAGAT GGACCAAAGC AGACCTCCCC AAGTAGAGAA GCTTCAGCTC | 700 |
| TGACAGCAGA GGGTGGAAGC TGCCTCATCT CAGGACTCCA GCCTGGCAAC | 750 |
| TCCTACTGGC TGCAGCTGCG CAGCGAACCT GATGGGATCT CCCTCGGTGG | 800 |
| CTCCTGGGGA TCCTGGTCCC TCCCTGTGAC TGTGGACCTG CCTGGAGATG | 850 |
| CAGTGGCACT TGGACTGCAA TGCTTTACCT TGGACCTGAA GAATGTTACC | 900 |
| TGTCAATGGC AGCAACAGGA CCATGCTAGC TCCCAAGGCT TCTTCTACCA | 950 |
| CAGCAGGGCA CGGTGCTGCC CCAGAGACAG GTACCCCATC TGGGAGAACT | 1000 |
| GCGAAGAGGA AGAGAAAACA AATCCAGGAC TACAGACCCC ACAGTTCTCT | 1050 |
| CGCTGCCACT TCAAGTCACG AAATGACAGC ATTATTCACA TCCTTGTGGA | 1100 |
| GGTGACCACA GCCCCGGGTA CTGTTCACAG CTACCTGGGC TCCCCTTTCT | 1150 |
| GGATCCACCA GGCTGTGCGC CTCCCCACCC CAAACTTGCA CTGGAGGGAG | 1200 |
| ATCTCCAGTG GGCATCTGGA ATTGGAGTGG CAGCACCCAT CGTCCTGGGC | 1250 |
| AGCCCAAGAG ACCTGTTATC AACTCCGATA CACAGGAGAA GGCCATCAGG | 1300 |
| ACTGGAAGGT GCTGGAGCCG CCTCTCGGGG CCCGAGGAGG GACCCTGGAG | 1350 |
| CTGCGCCCGC GATCTCGCTA CCGTTTACAG CTGCGCGCCA GGCTCAACGG | 1400 |
| CCCCACCTAC CAAGGTCCCT GGAGCTCGTG GTCGGACCCA ACTAGGGTGG | 1450 |
| 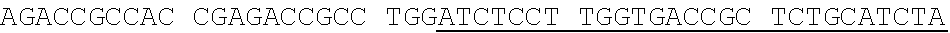 | 1500 |
| 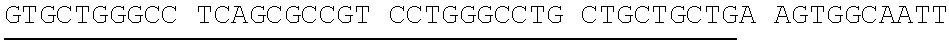 | 1550 |
| TCCTGCGCAC TACAGGAGAC TGAGGCATGC TTTGTGGCCC TCGCTTCCAG | 1600 |
| ACCTACACCG GGTCCTAGGC CAGTACCTCA GAGACACTGC AGCCCTAAGT | 1650 |
| CCTTCTAAGG CCACGGTTAC CGATAGCTGT GAAGAAGTGG AACCCAGCCT | 1700 |
| CCTGGAAATC CTCCCTAAGT CCTCAGAGAG CACTCCTTTA CCTCTGTGTC | 1750 |
| CCTCCCAACC TCAGATGGAC TACAGAGGAC TGCAACCTTG CCTGCGGACC | 1800 |
| ATGCCCCTGT CTGTGTGTCC ACCCATGGCT GAGACGGGGT CCTGCTGCAC | 1850 |
| CACACACATT GCCAACCACT CCTACCTACC ACTAAGCTAT TGGCAGCAGC | 1900 |
| CCTGA | 1905 |

The sequence encoding the human extracellular and transmembrane domain sequences are bolded, and the transmembrane domain is also underlined.

In preferred embodiments, the transgenic non-human mammal of the present invention has a baseline blood platelet count corresponding to a physiological blood platelet count of a matched non-transgenic non-human mammal, i.e., the transgenic non-human mammal has a baseline blood platelet count that falls within the same range as the baseline blood platelet count of a matched non-transgenic non-human mammal. The physiological blood platelet count of the matched non-transgenic mouse comprises a range of about 300×10³/μl to about 1600×10³/μl (see The Mouse in Biomedical Research, Fox et al., eds. Academic Press (2007); and Cheung et al., “Quantitative Trait Loci for Steady-State Platelet Count in Mice,” Mamm. Genome 15(10):784-97 (2004), which are hereby incorporated by reference in their entirety).

Another aspect of the present invention relates to isolated cells or tissue derived from the transgenic non-human mammals described above. These cells can be isolated from any tissues of the transgenic non-human mammal, but are preferably those cells that carry the transgene. Cells can be isolated using conventional cell harvesting techniques.

The present invention provides for transgenic animals that carry a humanized or a chimeric thrombopoietin receptor transgene in all their cells, as well as animals which carry the transgene in some, but not all their cells, i.e., mosaic animals. The transgene may also be selectively introduced into and activated in a particular cell type by following, for example, the teaching of Lasko et al., “Targeted Oncogene Activation by Site-Specific Recombination in Transgenic Mice,” Proc. Natl. Acad. Sci. USA 89: 6232-6236 (1992), which is hereby incorporated by reference in its entirety. The regulatory sequences required for such a cell-type specific activation will depend upon the particular cell type of interest, and will be apparent to those of skill in the art. When it is desired that the humanized or chimeric thrombopoietin receptor is integrated into the chromosomal site of the endogenous thrombopoietin receptor gene, gene targeting is preferred. Briefly, when such a technique is to be utilized, vectors containing some nucleotide sequences homologous to the endogenous thrombopoietin receptor gene are designed for the purpose of integrating, via homologous recombination with chromosomal sequences, into and disrupting the function of the nucleotide sequence of the endogenous thrombopoietin receptor gene.

The first step in the use of gene targeting to produce the transgenic animals of this invention is to prepare a DNA sequence carrying the transgene of interest, i.e., a “targeting molecule” or “targeting vector”. In one embodiment of the present invention, the targeting vector is capable of specifically disrupting an endogenous thrombopoietin receptor gene in transgenic animal cells carrying that gene and rendering that gene nonfunctional, while introducing a new or modified gene, e.g., the humanized or chimeric thrombopoietin receptor gene. In another embodiment of the present invention, the targeting vector is not designed to disrupt the endogenous thrombopoietin receptor gene.

Production of a DNA targeting molecule requires a DNA clone containing at least a portion of the thrombopoietin receptor gene or DNA clones containing sequences between which at least a portion of the thrombopoietin receptor target gene lies. Such DNA clones used for practice of the present invention may be obtained by a variety of means. For example, a suitable humanized thrombopoietin receptor targeting molecule and a suitable chimeric thrombopoietin receptor targeting molecule may be obtained by following the gene cloning methods described herein.

A DNA targeting molecule that is capable of disrupting a functional thrombopoietin receptor gene native in cells of the transgenic animal (and simultaneously introducing the functional humanized of chimeric TPO receptor) may be produced using information and processes well known in the art. Such a DNA targeting molecule is capable of integrating at a native thrombopoietin receptor gene locus (“target gene locus”) and disrupting the thrombopoietin receptor gene expression associated with that locus so that no expression of native thrombopoietin receptor protein is possible. These essential functions depend on two basic structural features of the targeting molecule.

The first structural feature of the targeting molecule is a pair of regions that are homologous to chosen regions of the target gene locus. That homology (in terms of both sequence identity and length) causes the targeting molecule to integrate by base pairing mechanisms (“homologous recombination”) at the site chosen in the target gene locus in transfected cells. The regions of homology between the target gene and the targeting molecule result in site-specific integration of the heterologous sequence.

The second structural feature of the targeting molecule is a disrupting sequence between the homologous regions. The disrupting sequence prevents expression of functional thrombopoietin receptor protein from the thrombopoietin receptor target gene following the replacement of a portion of that target gene by the integrated targeting molecule.

Properties of the targeting molecule that may be varied in the practice of the present invention include the lengths of the homologous regions, what regions of the target gene locus are to be duplicated as the homologous regions of the targeting molecule, the length of the disrupting sequence, the identity of the disrupting sequence, and what sequence of the target gene is to be replaced by the targeting molecule.

It should be noted that the target gene locus nucleotide sequences chosen for homology in the targeting molecule remains unchanged after integration of the targeting molecule. Those sequences of the target gene locus are merely replaced by the duplicate (homologous) sequences in the targeting molecule. Identity between the chosen regions of the target gene locus and the homologous regions in the targeting molecule is the means by which the targeting molecule delivers the disrupting sequence precisely into the thrombopoietin receptor target gene.

For some embodiments of the present invention it is preferred that the disrupting sequence have a dual function, i.e., be both a selectable marker and a disrupting sequence. In those embodiments, the length and identity of the disrupting sequence will be determined largely by the selectable marker coding sequence and associated expression control sequences. The selectable marker gene provides for positive selection of transfected cells that have taken up and integrated the targeting molecule. The need for a selectable marker will depend on the methods chosen for transfection of cells and transgenic animal production. The choice of those methods, in turn, will depend on the species of animal on which this invention is being practiced. For example, a preferred method for production of transgenic mice involves murine ES cells, and a preferred method of transfecting ES cells is electroporation, with which a selectable marker is preferred. The preferred selectable marker is the antibiotic resistance gene, neomycin phosphotransferase (“neo”). A neo gene with mammalian expression control sequences is commercially available (Stratagene Cloning Systems, La Jolla, Calif.). Although neo is preferred for mammalian cell selection, other marker genes, such as thymidine kinase, dihydrofolate reductase, hygromycin B phosphotransferase, xanthine-guanine phosphoribosyl transferase, adenosine deaminase, asparagine synthetase and CAD (carbamyl phosphate synthetase/aspartate transcarbamylase/dihydroorotase) may be used with appropriate culture media.

The targeting molecule can be a linear DNA molecule or a circular DNA molecule. A circular targeting molecule can comprise a pair of homologous regions separated by the transgene, as described for a linear targeting molecule. Alternatively, a circular targeting molecule can comprise a single homologous region. Upon integration at the target gene locus, the circular molecule would become linearized, with a portion of the homologous region at each end. Thus, the single homologous region effectively becomes two homologous regions, as described in the discussion of linear targeting molecules (see Watson et al., Molecular Biology of the Gene (4th Ed.), Benjamin/Cummings, Menlo Park, Calif., p. 606, which is hereby incorporated by reference in its entirety).

Once a DNA targeting molecule carrying the humanized or chimeric thrombopoietin receptor gene has been produced, it may be introduced into a desired animal cell to produce a founder line of the desired transgenic animals. The cell type chosen for transfection with the thrombopoietin targeting molecule must be pluripotent. The defining characteristic of pluripotent cells is developmental plasticity, which is necessary for production of a transgenic animal. Pluripotent cells are exemplified by oocytes, sperm and embryonic cells. Oocytes and embryonic cells are preferred in the practice of the present invention. Animal species is a major factor in the choice of pluripotent cell type to be used in practicing the present invention.

A DNA targeting molecule carrying the humanized or chimeric thrombopoietin receptor gene can be integrated into the genome of the founder line of transgenic animals using any standard method well known to those skilled in the art (see e.g., Hogan et al., Manipulating the Mouse Embryo: A Laboratory Manual (Cold Spring Harbor Laboratory, 1986); Hogan et al., Manipulating the Mouse Embryo: A Laboratory Manual (Cold Spring Harbor Laboratory, 1994), and U.S. Pat. No. 5,602,299 to Lazzarini; U.S. Pat. No. 5,175,384 to Krimpenfort; U.S. Pat. No. 6,066,778 to Ginsburg; and U.S. Pat. No. 6,037,521 to Sato et al, which are hereby incorporated by reference in their entirety). Such techniques include, but are not limited to, pronuclear microinjection (U.S. Pat. No. 4,873,191 to Wagner et al., which is hereby incorporated by reference in its entirety); retrovirus mediated gene transfer into germ lines (Van der Putten et al., Proc. Natl. Acad. Sci. USA 82:6148-6152 (1985), which is hereby incorporated by reference in its entirety); gene targeting in embryonic stem cells (Thompson et al., Cell 56:313-321 (1989), which is hereby incorporated by reference in its entirety); electroporation of embryos (Lo et al., Mol. Cell. Biol. 3:1803-1814 (1983), which is hereby incorporated by reference in its entirety); and sperm-mediated gene transfer (Lavitrano et al., Cell 57:717-723 (1989), which is hereby incorporated by reference in its entirety).

For example, embryonic cells at various developmental stages can be used to introduce transgenes for the production of transgenic animals. Different methods are used depending on the stage of development of the embryonic cell. The zygote is a good target for micro-injection, and methods of microinjecting zygotes are well known to (see U.S. Pat. No. 4,873,191 to Wagner et al., which is hereby incorporated by reference in its entirety). In the mouse, the male pronucleus reaches the size of approximately 20 micrometers in diameter which allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 (1985), which is hereby incorporated by reference in its entirety). As a consequence, all cells of the transgenic non-human animal will carry the incorporated transgene. This will in general also be reflected in the efficient transmission of the transgene to offspring of the founder since 50% of the germ cells will harbor the transgene.

The transgenic animals of the present invention can also be generated by introduction of the targeting vectors into embryonic stem (ES) cells. ES cells are obtained by culturing pre-implantation embryos in vitro under appropriate conditions (Evans et al., Nature 292:154-156 (1981); Bradley et al., Nature 309:255-258 (1984); Gossler et al., Proc. Natl. Acad. Sci. USA 83:9065-9069 (1986); and Robertson et al., Nature 322:445-448 (1986), which are hereby incorporated by reference in their entirety). Transgenes can be efficiently introduced into the ES cells by DNA transfection using a variety of methods known to the art including electroporation, calcium phosphate co-precipitation, protoplast or spheroplast fusion, lipofection and DEAE-dextran-mediated transfection. Transgenes can also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (reviewed in Jaenisch, Science 240:1468-1474 (1988), which is hereby incorporated by reference in its entirety). Prior to the introduction of transfected ES cells into the blastocoel, the transfected ES cells can be subjected to various selection protocols to enrich for ES cells that have integrated the transgene if the transgene provides a means for such selection. Alternatively, PCR can be used to screen for ES cells that have integrated the transgene. This technique obviates the need for growth of the transfected ES cells under appropriate selective conditions prior to transfer into the blastocoel.

In addition, retroviral infection can also be used to introduce transgenes into a non-human animal. The developing non-human embryo can be cultured in vitro to the blastocyst stage. During this time, the blastomeres can be targets for retroviral infection (Janenich, Proc. Natl. Acad. Sci. USA 73:1260-1264 (1976), which is hereby incorporated by reference in its entirety). The viral vector system used to introduce the transgene is typically a replication-defective retrovirus carrying the transgene (Jahner et al., Proc. Natl. Acad. Sci. USA 82:6927-6931 (1985); Van der Putten et al. Proc. Natl. Acad. Sci. USA 82:6148-6152 (1985)). Transfection is easily and efficiently obtained by culturing the blastomeres on a monolayer of virus-producing cells. Alternatively, infection can be performed at a later stage. Additional means of using retroviruses or retroviral vectors to create transgenic animals known to the art involves the micro-injection of retroviral particles or mitomycin C-treated cells producing retrovirus into the perivitelline space of fertilized eggs or early embryos (WO 90/08832 to Onions, which is hereby incorporated by reference in its entirety).

The present invention provides transgenic non-human animals that carry the transgene in all their cells, as well as animals that carry the transgene in some, but not all their cells, i.e., expression of the transgene is controlled by a cell specific promoter and/or enhancer elements placed upstream of the transgene. Expression or cloning constructs suitable for driving transgene expression in a transgenic animal are well known in the art. Other components of the expression construct include a strong polyadenylation site, appropriate restriction endonuclease sites, and introns to ensure the transcript is spliced.

Both of the human TPO receptor (c-Mpl) knock-in mouse models described herein have utility in screening and identifying TPO mimetics having clinical relevance and efficacy for a number of human conditions including, without limitation, (i) thrombocytopenia of various etiology such as autoimmune related bone marrow pathology, viral related bone marrow pathology, radiation induced bone marrow injury, and chemotherapy induced bone marrow injury; (ii) abnormal hematopoiesis caused by bone marrow abnormality, such as autoimmune related bone marrow pathology, viral related bone marrow pathology, radiation induced bone marrow injury, and chemotherapy induced bone marrow injury; (iii) hematopoietic stem cell function, stem cell and tissue repair/regeneration through c-mpl receptor mediated mechanisms, stem cells and organ repair/regeneration through c-mpl receptor mediated mechanisms; and (iv) vascular niche formation.

Candidate TPO mimetics, TPO receptor agonists, and TPO receptor antagonist compounds can be screened using the non-human transgenic mammals of the present invention comprising a humanized or chimeric TPO receptor using methods readily known in the art. For example, the Mpl^{hExon10} knock-in and the Mpl^{hmMpl} knock-in transgenic mouse models described herein is particularly suitable for TPO mimetic, agonist, and antagonist screening. In a typical screening assay, a candidate compound is administered to the transgenic animal, e.g., by gavage. In one embodiment, administration of the candidate compound can be carried out daily for 5, 10, 15, 20, 25, days or longer. Preferably, a range of doses of the candidate compound are administered, e.g., 3 mg/kg, 5 mg/kg, 10 mg/kg, 15 mg/kg, etc. Serial blood samples are analyzed for blood counts and other desired endpoints (e.g., TPO mimetic metabolism) in both pre- and post-administration collected samples. Preferably, the post-administration blood samples are taken at regular intervals following administration and for some time-period following the final administration (e.g., 1, 5, 7, 10, 20, 30-days) to collect pharmacokinetic data. The measured blood counts or other desired endpoints in post-administration samples are compared to corresponding measurements in a reference sample, e.g., pre-administration sample from the same animal, or a sample from a transgenic control animal that was not administered the candidate compound, to identify whether the candidate compound is a TPO mimetic, TPO receptor agonist, or TPO receptor antagonist.

A number of endpoints can be measured when using the transgenic non-human animal to test the efficacy of TPO mimetic compounds. These endpoints include, without limitation, cell proliferation, cell differentiation, and gene expression in one or more cell types or tissues of the transgenic non-human mammal; cell repair, tissue repair and/or regeneration, and organ repair and/or regeneration in one or more cell types, tissues, or organs of the transgenic non-human mammal. Particular cell types to assess for such endpoints include, without limitation, platelets, megakaryocytes, red blood cells, white blood cells, hematopoietic stem cells, bone marrow progenitor and precursor cells, and precursor/progenitor cells for thrombopoiesis. Particular tissues/organs include, without limitation, intestine, esophagus, stomach, colon, rectum, lung, trachea and bronchus, bone, cartilage, heart, muscle, tendon, skin, hair follicle, nerves, brain, spinal cord, liver, pancreas, kidney, skin, vessels, blood, bone marrow, lymph node, eye, ear, adipose tissue, connective tissue, salivary gland, an exocrine organ, or an endocrine organ.

In one embodiment of the present invention, the platelet blood count is measured after administration of a candidate compound. An increase in the measured blood platelet count after administration compared to the reference blood platelet count indicates that the candidate compound is a human thrombopoietin mimetic or human thrombopoietin receptor agonist. Thrombocytopenia may be induced in the transgenic non-human animal prior to screening the candidate compound. Thrombocytopenia can be induced by an autoimmune condition, a viral infection, radiation exposure, chemotherapy, or a combination thereof.

In another embodiment of the present invention, hematopoietic stem cell (HSC) count or a bone marrow progenitor/precursor cell count of one or more lineages is measured after administration of a candidate compound. An increase in the obtained HSC count or bone marrow progenitor/precursor cell count compared to the reference HSC or bone marrow progenitor/precursor cell count (e.g., HSC or bone marrow progenitor count pre-administration) indicates the candidate compound is a human thrombopoietin mimetic or human thrombopoietin receptor agonist. Abnormal hematopoiesis can be induced in the transgenic non-human mammal prior to administering the candidate compound for screening.

In another embodiment of the present invention, traumatic injury, radiation injury, chemical injury, infectious agent injury, or a combination thereof can be induced in the transgenic non-human mammal prior to screening candidate compounds to identify TPO mimetics, receptor agonists, and/or receptor antagonists. In another embodiment of the present invention, the transgenic non-human mammal can have a congenital defect.

Candidate TPO mimetic compounds can also be screened using isolated cells or tissue derived from the transgenic non-human mammal of the present invention. The isolated cells can be obtained from the transgenic non-human mammal using standard tissue harvesting techniques to allow for the recovery of cells. Suitable tissues that can be harvested include, without limitation, intestine, esophagus, stomach, colon, rectum, lung, trachea and bronchus, bone, cartilage, heart, muscle, tendon, skin, hair follicle, nerves, brain, spinal cord, liver, pancreas, kidney, skin, vessels, blood, bone marrow, lymph node, eye, ear, adipose tissue, connective tissue, salivary gland, an exocrine organ, or an endocrine organ. After treating the isolated cells, one or more endpoints can be evaluated to assess the effects of the treatment. These endpoints include, without limitation, cell proliferation level, cell differentiation level, and gene expression level in the isolated cells or tissue.

When the one or more measured endpoints is cell proliferation, and an increase in the level of cell proliferation in the isolated cells or tissue administered the candidate compound is observed compared to the level of cell proliferation in the reference sample, the candidate compound is a thrombopoietin mimetic or thrombopoietin receptor agonist. Likewise, when the one or more measured endpoints is cell differentiation, and an increase in the level of cell differentiation in the isolated cells or tissue administered the candidate compound is observed compared to the level of cell differentiation in the reference sample, the candidate compound is a thrombopoietin mimetic or thrombopoietin receptor agonist.

Suitable TPO agonists that can be screened using the transgenic non-human mammals of the present invention include, without limitation, non-peptide and peptide thromobopoietin mimetics, agonist antibodies, peptibodies, and small molecules.

Based on the data presented in the accompanying examples, it is believed that c-Mpl receptor agonists are useful for several therapeutic uses that could not have been demonstrated previously without the benefit of the transgenic non-human models described herein.

One aspect of the present invention is directed to a method of treating a subject for acute radiation syndrome (ARS) that involves administering a c-Mpl receptor agonist to the subject under conditions effective to treat acute radiation syndrome. ARS, which is also known as radiation poisoning, radiation sickness or radiation toxicity, can result from exposure to external radiation, internal radiation (e.g., inhalation, injection, or ingestion), or both. ARS arises when a subject receives a non-therapeutically high dose of radiation, typically to the whole body or majority of the body, over a short period of time, usually within minutes. In one embodiment of the present invention, the subject has radiation hematopoietic syndrome. In accordance with this aspect of the present invention, the c-Mpl receptor agonist can be administered prior to and/or after the exposure to radiation for purposes of treating ARS and its clinical manifestations. Administration of a suitable c-Mpl agonist is repeated as necessary to treat ARS and its clinical manifestations, which include, without limitation, radiation hematopoietic syndrome, gastrointestinal syndrome, and cerebrovascular syndrome.

Another aspect of the present invention is directed to a method of treating a subject for chronic radiation syndrome that involves administering a c-Mpl receptor agonist to the subject under conditions effective to treat chronic radiation syndrome. Chronic radiation syndrome, also known as delayed effects of acute radiation exposure (DEARE), encompasses a variety of health effects that occur after months or years of chronic, repeated exposure to high amounts of radiation.

Another aspect of the present invention is directed to a method of treating a subject having a bone marrow injury resulting from exposure to a non-therapeutic chemical agent. This method involves administering a c-Mpl receptor agonist to the subject under conditions effective to treat the bone marrow injury resulting from exposure to non-therapeutic chemical agent. Non-therapeutic chemical agents that cause bone marrow toxicity or injury include, without limitation, 2,2,-dichlordiethyl sulfide (mustard gas), pinacolyl methylphosphono-fluoridate (soman; nerve gas), and nitrogen mustard. In accordance with this aspect of the present invention, a suitable subject is one that has been exposed or is at risk of being exposed to a non-therapeutic chemical. When a subject is at risk for such exposure, the c-Mpl receptor agonist can be administered prior to exposure and repeated as necessary after the exposure to effectuate treatment.

Yet another aspect of the present invention relates to a method of inducing tissue repair or tissue regeneration in a subject that includes administering a c-Mpl receptor agonist to the subject under conditions effective to induce tissue repair or tissue regeneration in the subject. Tissues that can be repaired or regenerated include, without limitation, cells or tissue of intestine, esophagus, stomach, colon, rectum, lung, trachea, bronchus, bone, cartilage, heart, muscle, tendon, skin, hair follicle, nerves, brain, spinal cord, liver, pancreas, kidney, spleen, blood vessels, bone marrow, lymph node, eyes, ears, adipose tissue, connective tissue, salivary gland, an exocrine organ, or an endocrine organ. Subjects suitable for treatment in accordance with this aspect of the present invention include subjects having a condition that causes tissue or cell degeneration or death, including, for example, myocardial infarction, vascular injury, stroke, spinal cord injury, an infectious disease, an autoimmune disorder, acute or chronic radiation syndrome, a congenital condition, and the aging process.

In accordance with these aspects of the present invention, suitable c-Mpl receptor agonists include, without limitation, recombinant thrombopoietin (TPO) protein or peptide fragment thereof, non-peptide thrombopoietin mimetics, thrombopoietin peptide mimetics and peptibodies, and c-Mpl receptor agonist antibodies (see Kuter D J, “New Thrombopoietic Growth Factors,” Blood 109(11):4607-4616 (2007), which is hereby incorporated by reference in its entirety) as described in more detail below. The c-Mpl receptor agonist can be administered in combination with a cell therapy, one or more cytokines (e.g., G-CSF, GM-CSF, thrombopoietin, M-CSF, erythropoietin, Gro-beta, IL-11, SCF, FLT3 ligand, LIF, IL-3, IL-6, IL-1, progenipoietin, NESP, SD-01, IL-5, VEGF, FGF, KGF or any combination thereof), and one or more immune modulators (e.g., SCV-07, Glatiramer acetate, or a combination thereof). The cytokine and/or immune modulator can be administered prior to, concurrently with, or after administering the c-Mpl receptor agonist.

In one embodiment of the present invention, the c-Mpl receptor agonist is a small molecule non-peptide TPO mimetic. Suitable non-peptide TPO mimetics include, without limitation, hydroxyl-1-azo-benzene, such as those disclosed in U.S. Pat. No. 7,160,870 to Duffy et al., which is hereby incorporated by reference in its entirety. Suitable hydroxyl-1-azo-benzene derivatives include compounds of Formula (I):

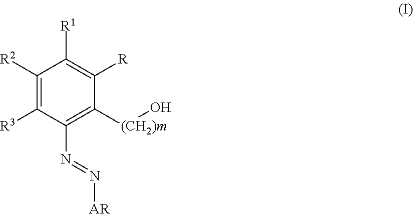

wherein,
R, R¹, R² and R³ are each independently selected from hydrogen, C₁₋₆alkyl, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid, —SO₂NR⁵R⁶, and a heterocyclic methylene substituent as represented by Formula (III),

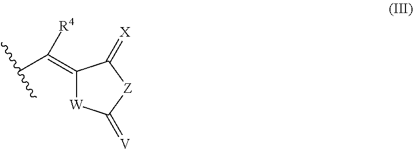

where,
p is 0-6,
n is 0-2,
V, W, X and Z are each independently selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R⁴ is selected from: hydrogen, alkyl, cycloalkyl, C₁-C₁₂aryl, substituted alkyl, substituted cycloalkyl and substituted C₁-C₁₂aryl, and
R⁵ and R⁶ are each independently selected from hydrogen, alkyl, substituted alkyl, C₃₋₆ cycloalkyl, and aryl, or R⁵ and R⁶ taken together with the nitrogen to which they are attached represent a 5 to 6 member saturated ring containing up to one other heteroatom selected from oxygen and nitrogen;
m is 0-6; and
AR is a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, aryl, substituted cycloalkyl, substituted aryl, aryloxy, oxo, hydroxy, alkoxy, cycloalkyl, acyloxy, amino, N-acylamino, nitro, cyano, halogen, —C(O)OR⁴, —C(O)NR¹⁰R¹¹, —S(O)₂NR¹⁰R¹¹, —S(O)ₙR⁴, and protected —OH,

where n is 0-2,
R⁴ is hydrogen, alkyl, cycloalkyl, C₁-C₁₂aryl, substituted alkyl, substituted cycloalkyl and substituted C₁-C₁₂aryl, and
R¹⁰ and R¹¹ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR⁴, —S(O)ₙR⁴, —C(O)NR⁴R⁴, —S(O)₂NR⁴R⁴, nitro, cyano, cycloalkyl, substituted cycloalkyl, halogen, aryl, substituted aryl and protected —OH,
or R¹⁰ and R¹¹ taken together with the nitrogen to which they are attached represent a 5 to 6 member saturated ring containing up to one other heteroatom selected from oxygen and nitrogen, where R⁴ is as described above and n is 0-2; and
   pharmaceutically acceptable salts, hydrates, solvates and esters thereof; provided that at least one of R, R¹, R² and R³ is a substituted aryl group or a heterocyclic methylene substituent as represented in Formula (III).

One class of compounds of Formula (I) above includes compounds having Formula (V)

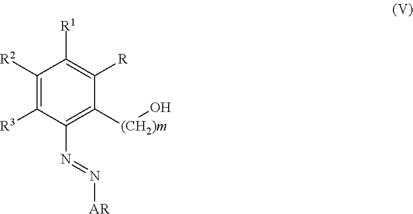

R, R¹, R² and R³ are each independently selected from hydrogen, C₁₋₆alkyl, C₁₋₆alkoxy, (CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid and —SO₂NR⁵R⁶,

where,
p is 0-6,
n is 0-2,
R⁴ is selected from: hydrogen, alkyl, cycloalkyl, C₁-C₁₂aryl, substituted alkyl, substituted cycloalkyl and substituted C₁-C₁₂ aryl, and
R⁵ and R⁶ are each independently selected from hydrogen, alkyl, substituted alkyl, C₃₋₆ cycloalkyl, and aryl,
or R⁵ and R⁶ taken together with the nitrogen to which they are attached represent a 5 to 6 member saturated ring containing up to one other heteroatom selected from oxygen and nitrogen;
m is 0-6; and
AR is a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, aryl, substituted cycloalkyl, substituted aryl, aryloxy, oxo, hydroxy, alkoxy, cycloalkyl, acyloxy, amino, N-acylamino, nitro, cyano, halogen, —C(O)OR⁴, —C(O)NR¹⁰R¹¹, —S(O)₂NR¹⁰R¹¹, —S(O)ₙR⁴ and protected —OH,

where n is 0-2,
R⁴ is hydrogen, alkyl, cycloalkyl, C₁-C₁₂aryl, substituted alkyl, substituted cycloalkyl and substituted C₁-C₁₂aryl; and
R¹⁰ and R¹¹ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR⁴, —S(O)ₙR⁴, —C(O)NR⁴R⁴, —S(O)₂NR⁴R⁴, nitro, cyano, cycloalkyl, substituted cycloalkyl, halogen, aryl, substituted aryl and protected —OH,
or R¹⁰ and R¹¹ taken together with the nitrogen to which they are attached represent a 5 to 6 member saturated ring containing up to one other heteroatom selected from oxygen and nitrogen,
where R⁴ is as described above and n is 0-2; and
   pharmaceutically acceptable salts, hydrates, solvates and esters thereof; provided that at least one of R, R¹, R² and R³ is a substituted aryl group.

Another class of compounds of Formula (I) includes compounds of Formula (II)

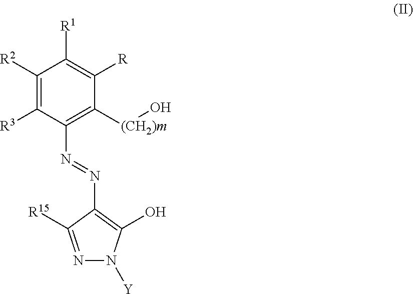

R, R¹, R² and R³ are each independently selected from hydrogen, C₁₋₆alkyl, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid, —SO₂NR⁵R⁶, and a heterocyclic methylene substituent as represented by Formula (III),

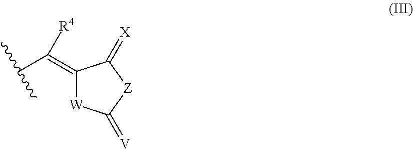

where
p is 0-6,
n is 0-2,
V, W, X and Z are each independently selected from O, S, and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R⁴ is hydrogen, alkyl, cycloalkyl, C₁-C₁₂aryl, substituted alkyl, substituted cycloalkyl and substituted C₁-C₁₂aryl, and
R⁵ and R⁶ are each independently selected from hydrogen, alkyl, substituted alkyl, C₃₋₆cycloalkyl, and aryl,
or R⁵ and R⁶ taken together with the nitrogen to which they are attached represent a 5 to 6 member saturated ring containing up to one other heteroatom selected from oxygen and nitrogen;
R¹⁵ is selected from the group consisting of alkyl, C₁-C₁₂aryl, hydroxy, alkoxy, substituted alkyl, substituted C₁-C₁₂aryl and halogen;
m is 0-6; and
Y is selected from alkyl, substituted alkyl and a cyclic or polycyclic aromatic ring containing from 3 to 14 carbon atoms and optionally containing from one to three heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, C₁-C₁₂aryl, substituted cycloalkyl, substituted C₁-C₁₂aryl, hydroxy, aryloxy, alkoxy, cycloalkyl, nitro, cyano, halogen and protected —OH; and
   pharmaceutically acceptable salts, hydrates, solvates and esters thereof;

provided that at least one of R, R¹, R² and R³ is a substituted aryl group or a heterocyclic methylene substituent as represented in Formula (III).

Included among the compounds of Formula (II) are those having Formula (VI):

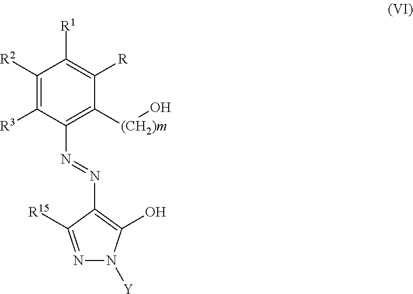

where
R, R¹, R² and R³ are each independently selected from hydrogen, C₁₋₆alkyl, C₁₋₆ alkoxy, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid and —SO₂NR⁵R⁶,

where
p is 0-6,
n is 0-2,
R⁴ is hydrogen, alkyl, cycloalkyl, C₁-C₁₂aryl, substituted alkyl, substituted cycloalkyl and substituted C₁-C₁₂aryl, and

R⁵ and R⁶ are each independently selected from hydrogen, alkyl, substituted alkyl, C₃₋₆cycloalkyl, and aryl,
or R⁵ and R⁶ taken together with the nitrogen to which they are attached represent a 5 to 6 member saturated ring containing up to one other heteroatom selected from oxygen and nitrogen;
R¹⁵ is selected from the group consisting of alkyl, C₁-C₁₂aryl, hydroxy, alkoxy, substituted alkyl, substituted C₁-C₁₂aryl and halogen;
m is 0-6; and
Y is selected from alkyl, substituted alkyl and a cyclic or polycyclic aromatic ring containing from 3 to 14 carbon atoms and optionally containing from one to three heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, C₁-C₁₂aryl, substituted cycloalkyl, substituted C₁-C₁₂aryl, hydroxy, aryloxy, alkoxy, cycloalkyl, nitro, cyano, halogen and protected —OH; and
   pharmaceutically acceptable salts, hydrates, solvates and esters thereof;

Exemplary hydroxyl-1-azo-benzene compounds of the present invention include, without limitation:
4′-{N′-[1-(3,4-Dimethylphenyl)-3-methyl-5-oxo-1,5-dihydropyrazol-4-ylidene]hydrazino}-3′-hydroxybiphenyl-4-carboxylic acid;
4′-{N′-[1-(3,4-Dimethylphenyl)-3-methyl-5-oxo-1,5-dihydropyrazol-4-ylidene]hydrazino}-3′-hydroxybiphenyl-3-carboxylic acid;
3′-{N′-[1-(3,4-Dimethylphenyl)-3-methyl-5-oxo-1,5-dihydropyrazol-4-ylidene]hydrazino}-2′-hydroxybiphenyl-3-carboxylic acid;
3′-{N′-[1-(4-tert-Buthylphenyl)-3-methyl-5-oxo-1,5-dihydropyrazol-4-ylidene]hydrazino}-2′-hydroxybiphenyl-3-carboxylic acid;
2-Aza-3′-{N′-[1-(4-tert-butylphenyl)-3-methyl-5-oxo-1,5-dihydropyrazol-4-ylidene]hydrazino}-5′-chloro-2′-hydroxybiphenyl-3-carboxylic acid;
2-Aza-3′-{N′-[1-(4-tert-butylphenyl)-3-methyl-5-oxo-1,5-dihydropyrazol-4-ylidene]hydrazino}-2′-hydroxybiphenyl-3-carboxylic acid;
3-Aza-3′-{N′-[1-(4-tert-butylphenyl)-3-methyl-5-oxo-1,5-dihydropyrazol-4-ylidene]hydrazino}-2′-hydroxybiphenyl-5-carboxylic acid;
2-Aza-5′-chloro-3′-{N′-[1-(3,4-dimethylphenyl)-3-methyl-5-oxo-1,5-dihydropyrazol-4-ylidene]hydrazino}-2′-hydroxybiphenyl-3-carboxylic acid;
2-Aza-3′-{N′-[1-(4-tert-butylphenyl)-3-methyl-5-oxo-1,5-dihydropyrazol-4-ylidene]hydrazino}-2′-hydroxy-5′-methylbiphenyl-3-carboxylic acid;
2-Aza-3′-{N′-[1-(3,4-dimethylphenyl)-3-methyl-5-oxo-1,5-dihydropyrazol-4-ylidene]hydrazino}-2′-hydroxy-5′-methylbiphenyl-3-carboxylic acid;
3′-{N′-[1-(4-tert-Butylphenyl)-3-methyl-5-oxo-1,5-dihydropyrazol-4-ylidene]hydrazino}-2′-hydroxy-5′-methylbiphenyl-3-carboxylic acid;
3-{N′-[1-(3,4-dimethylphenyl)-3-methyl-5-oxo-1,5-dihydropyrazol-4-ylidene]hydrazino}-2-hydroxy-3′-tetrazol-5-ylbiphenyl;
3′-{N′-[1-(3,4-dimethylphenyl)-3-methyl-5-oxo-1,5-dihydropyrazol-4-ylidene]hydrazino}-5′-fluoro-2′-hydroxybiphenyl-3-carboxylic acid;
7-({N′-[1-(3,4-dimethylphenyl)-3-methyl-5-oxo-1,5-dihydropyrazol-4-ylidene]hydrazino}-2-hydroxyphenyl)quinolin-4[1H]-one-3-carboxylic acid;
7-({N′-[1-(4-tert-butylphenyl)-3-methyl-5-oxo-1,5-dihydropyrazol-4-ylidene]hydrazino}-2-hydroxyphenyl)quinolin-4[1H]-one-3-carboxylic acid;
3-Aza-3′-{N′-[1-(3,4-dimethylphenyl)-3-methyl-5-oxo-1,5-dihydropyrazol-4-ylidene]hydrazino}-2′-hydroxybiphenyl-5-carboxylic acid;
3-Aza-3′-(N′-[1-{3-methyl-[4-(1-methylethyl)phenyl]-5-oxo-1,5-dihydropyrazol-4-ylidene}hydrazino)-2′-hydroxybiphenyl-5-carboxylic acid;
3-Aza-3′-{N′-[1-(4-tertbutylphenyl-3-methyl-5-oxo-1,5-dihydropyrazol-4-ylidene]hydrazino}-2′-hydroxybiphenyl-5-carboxylic acid;
5′-Chloro-3′-{N′-[1-(3,4-dimethylphenyl)-3-methyl-5-oxo-1,5-dihydropyrazol-4-ylidene]hydrazino}-2′-hydroxybiphenyl-3-carboxylic acid;
3′-{N′-[1-(3,4-Dimethylphenyl)-3,5-dioxo-1,5-dihydropyrazol-4-ylidene]hydrazino}-2′-hydroxybiphenyl-3-carboxylic acid;
3′-{N′-[1-(2-Ethoxy-2-oxoethyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-2-hydroxy-4′-(tetrazol-5-yl)biphenyl;
3′-{N′-{1-[2-(N-tert-butyl)amino-2-oxoethyl]-3-methyl-5-oxo-1,5-dihydropyrazol-4-ylidene}hydrazino}-2′-hydroxybiphenyl-3-carboxylic acid;
3′-{N′-[3-Chloro-1-(3,4-dimethylphenyl)-5-oxo-1,5-dihydropyrazol-4-ylidene]hydrazino}-2′-hydroxybiphenyl-3-carboxylic acid;
5-chloro-3-{N′-[1-(3,4-dimethylphenyl)-3-methyl-5-oxo-1,5-dihydropyrazol-4-ylidene]hydrazino}-2-hydroxy-4′-(tetrazol-5-yl)biphenyl;
3′-{N′-[1-(3,4-Dimethylphenyl)-3-methyl-5-oxo-1,5-dihydropyrazol-4-ylidene]hydrazino}-2′-hydroxybiphenyl-3,5-dicarboxylic acid;
3-Aza-3′-{N′-[1-(3,4-dimethylphenyl)-3-methyl-5-oxo-1,5-dihydropyrazol-4-ylidene]hydrazino}-2′-hydroxy-5′-methylbiphenyl-5-carboxylic acid;
3′-{N′-[1-(3,4-Dimethylphenyl)-3-methyl-5-oxo-1,5-dihydropyrazol-4-ylidene]hydrazino}-2′-hydroxybiphenyl-4-carboxylic acid;
3′-{N′-[1-(3,4-Dimethylphenyl)-3-methoxy-5-oxo-1,5-dihydropyrazol-4-ylidene]hydrazino}-2′-hydroxybiphenyl-3-carboxylic acid;
3′-{N′-[1-(4-methoxyphenyl)-3-methyl-5-oxo-1,5-dihydropyrazol-4-ylidene]hydrazino}-2-hydroxybiphenyl-3-carboxylic acid;
(3-{N′-[1-(3,4-dimethylphenyl)-3-methyl-5-oxo-1,5-dihydropyrazol-4-ylidene]hydrazino}-2-hydroxy-3′-biphenyl)-1,1,1,-trifluoromethanesulfonamide;
3′-{N′-[1-(3,4-Dichlorophenyl)-3-methyl-5-oxo-1,5-dihydropyrazol-4-ylidene]hydrazino}-2′-hydroxybiphenyl-3-carboxylic acid;
3′-{N′-[3-methyl-5-oxo-1-(3-trifluoromethylphenyl)-1,5-dihydropyrazol-4-ylidene]hydrazino}-2′-hydroxybiphenyl-3-carboxylic acid;
8-{N′-[1-(3,4-dimethylphenyl)-3-methyl-5-oxo-1,5-dihydropyrazol-4-ylidene]hydrazino}-quinolin-4[1H]-one-3-carboxylic acid;
3′-{N′-[3-methyl-5-oxo-1-(4-trifluoromethylphenyl)-1,5-dihydropyrazol-4-ylidene]hydrazino}-2′-hydroxybiphenyl-3-carboxylic acid;
3′-{N′-[3-methyl-5-oxo-1-(4-N-methylcarboxamidolphenyl)-1,5-dihydropyrazol-4-ylidene]hydrazino}-2′-hydroxybiphenyl-3-carboxylic acid;
N-[1-(3′-{N′-[1-(3,4-dimethylphenyl)-3-methyl-5-oxo-1,5-dihydropyrazol-4-ylidene]hydrazino}-2′-hydroxybiphenyl-3-yl)methanoyl]methanesulfonamide;
3′-{N′-[3-methyl-5-oxo-1-phenyl-1,5-dihydropyrazol-4-ylidene]hydrazino}-2′-hydroxybiphenyl-3-carboxylic acid;
3′-{N′-[3-methyl-1-(4-methylphenyl)-5-oxo-1,5-dihydropyrazol-4-ylidene]hydrazino}-2′-hydroxybiphenyl-3-carboxylic acid;
3′-{N′-[1-(4-chlorophenyl)-3-methyl-5-oxo-1,5-dihydropyrazol-4-ylidene]hydrazino}-2′-hydroxybiphenyl-3-carboxyic acid;
3′-{N′-[1-(4-fluorophenyl)-3-methyl-5-oxo-1,5-dihydropyrazol-4-ylidene]hydrazino}-2′-hydroxybiphenyl-3-carboxylic acid;
3′-{N′-[3-methyl-5-oxo-1-(4-trifluoromethoxyphenyl)-1,5-dihydropyrazol-4-ylidene]hydrazino}-2′-hydroxybiphenyl-3-carboxylic acid;
3′-{N′-[1-(3,4-dimethylphenyl)-3-ethoxy-5-oxo-1,5-dihydropyrazol-4-ylidene]hydrazino}-2′-hydroxybiphenyl-3-carboxylic acid;
3′-{N′-[1-(3,4-dimethylphenyl)-3-(1-methylethoxy)-5-oxo-1,5-dihydropyrazol-4-ylidene]hydrazino}-2′-hydroxybiphenyl-3-carboxylic acid;
3′-{N′-[3-tert-butyl-1-(3,4-dimethylphenyl)-5-oxo-1,5-dihydropyrazol-4-ylidene]hydrazino}-2′-hydroxybiphenyl-3-carboxylic acid;
3′-{N′-[3-methyl-1-(4-methyl-2,3,5,6-tetrafluorophenyl)-5-oxo-1,5-dihydropyrazol-4-ylidene]hydrazino}-2′-hydroxybiphenyl-3-carboxylic acid;
3′-{N′-[1-(4-fluoro-3-methylphenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-phenyl-5-oxo-1,5-dihydropyrazol-4-ylidene]hydrazino}-2′-hydroxybiphenyl-3-carboxylic acid;
3-{N′-[1-(3,4-dimethylphenyl)-5-oxo-3-phenyl-1,5-dihydropyrazol-4-ylidene]hydrazino}-2-hydroxy-3′-tetrazol-5-ylbiphenyl;
3-{N′-[1-(3,4-dimethylphenyl)-3-methoxy-5-oxo-1,5-dihydropyrazol-4-ylidene]hydrazino}-2-hydroxy-3′-tetrazol-5-ylbiphenyl;
3-{N′-[1-(3,4-dimethylphenyl)-3-ethoxy-5-oxo-1,5-dihydropyrazol-4-ylidene]hydrazino}-2-hydroxy-3′-tetrazol-5-ylbiphenyl;
3-{N′-[1-(3,4-dimethylphenyl)-3-(1-methylethoxy)-5-oxo-1,5-dihydropyrazol-4-ylidene]hydrazino}-2-hydroxy-3′-tetrazol-5-ylbiphenyl;
3-{N′-[1-(4-fluorophenyl)-3-methyl-5-oxo-1,5-dihydropyrazol-4-ylidene]hydrazino}-2-hydroxy-3′-tetrazol-5-ylbiphenyl;
3-{N′-[1-(4-fluoro-3-methylphenyl)-3-methyl-5-oxo-1,5-dihydropyrazol-4-ylidene]hydrazino}-2-hydroxy-3′-tetrazol-5-ylbiphenyl;
3-{N′-[3-methyl-5-oxo-1-(4-trifluoromethylphenyl)-1,5-dihydropyrazol-4-ylidene]hydrazino}-2-hydroxy-3′-tetrazol-5-ylbiphenyl;
3′-{N′-[1-(3,4-dimethylphenyl)-3-(pyridin-4-yl-5-oxo-1,5-dihydropyrazol-4-ylidene]hydrazino}-2′-hydroxybiphenyl-3-carboxylic acid;
3-{N′-[1-(3,4-dimethylphenyl)-3-pyridin-4-yl-5-oxo-1,5-dihydropyrazol-4-ylidene]hydrazino}-2-hydroxy-3′-tetrazol-5-ylbiphenyl;
3-{N′-[1-(3,4-dimethylphenyl)-3-pyridin-2-yl-5-oxo-1,5-dihydropyrazol-4-ylidene]hydrazino}-2-hydroxy-3′-tetrazol-5-ylbiphenyl;
3′-{N′-[1-(3,4-dimethylphenyl)-3-(pyridin-2-yl-5-oxo-1,5-dihydropyrazol-4-ylidene]hydrazino}-2′-hydroxybiphenyl-3-carboxylic acid;
3-{N′-[1-(3-fluoro-4-methylphenyl)-3-methyl-5-oxo-1,5-dihydropyrazol-4-ylidene]hydrazino}-2-hydroxy-3′-tetrazol-5-ylbiphenyl;
3′-{N′-[1-(3-fluoro-4-methylphenyl)-3-methyl-5-oxo-1,5-dihydropyrazol-4-ylidene]hydrazino}-2′-hydroxybiphenyl-3-carboxylic acid;
3′-{N′-[3-methyl-5-oxo-1-(4-trifluoromethylpyrimidin-2-yl)-1,5-dihydropyrazol-4-ylidene]hydrazino}-2′-hydroxybiphenyl-3-carboxylic acid;
3′-N-tert-butoxycarbonylamino-3-{N′-[1-(3,4-Dimethylphenyl)-3-methyl-5-oxo-1,5-dihydropyrazol-4-ylidene]hydrazino}-2-hydroxybiphenyl;
3′-amino-3-{N′-[1-(3,4-dimethylphenyl)-3-methyl-5-oxo-1,5-dihydropyrazol-4-ylidene]hydrazino}-2-hydroxybiphenyl;
3-{N′-[1-(3-fluorophenyl)-3-methyl-5-oxo-1,5-dihydropyrazol-4-ylidene]hydrazino}-2-hydroxy-3′-tetrazol-5-ylbiphenyl;
3′-{N′-[1-(3-fluorophenyl)-3-methyl-5-oxo-1,5-dihydropyrazol-4-ylidene]hydrazino}-2-hydroxybiphenyl-3-carboxylic acid;
3-{N′[3-methyl-5-oxo-1-(2,3,4,5,6-pentafluorophenyl)-1,5-dihydropyrazol-4-ylidene]hydrazino}-2-hydroxy-3′-tetrazol-5-ylbiphenyl;
3′-{N′-[3-methyl-5-oxo-1-(2,3,4,5,6-pentafluorophenyl)-1,5-dihydropyrazol-4-ylidene]hydrazino}-2′-hydroxybiphenyl-3-carboxylic acid;
3′-{N′-[1-(3,4-difluorophenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methoxymethyl-5-oxo-1,5-dihydropyrazol-4-ylidene]hydrazino}-2′-hydroxybiphenyl-3-carboxylic acid;
3-{N′-[1-(3,4-dimethylphenyl)-3-methoxymethyl-5-oxo-1,5-dihydropyrazol-4-ylidene]hydrazino}-2-hydroxy-3′-tetrazol-5-ylbiphenyl;
3-{N′-[1-(3,4-difluorophenyl)-3-methyl-5-oxo-1,5-dihydropyrazol-4-ylidene]hydrazino}-2-hydroxy-3′-tetrazol-5-ylbiphenyl;
3′-{N′-[1-(3,4-dimethylphenyl)-5-oxo-3-trifluoromethyl-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-6-fluoro-2′-hydroxybiphenyl-3-carboxylic acid;
3′-{N′-[1-(3,4-dimethylphenyl)-5-oxo-3-propyl-1,5-dihydropyrazol-4-ylidene]hydrazino}-2′-hydroxybiphenyl-3-carboxylic acid;
3-{N′-[1-(3,4-dimethylphenyl)-5-oxo-3-propyl-1,5-dihydropyrazol-4-ylidene]hydrazino}-2-hydroxy-3′-tetrazol-5-ylbiphenyl;
3′-{N′-[1-(3,4-dimethylphenyl)-3-(1-methyl-1H-pyrrol-3-yl)-5-oxo-1,5-dihydropyrazol-4-ylidene]hydrazino}-2′-hydroxybiphenyl-3-carboxylic acid;
3-{N′-[1-(3,4-dimethylphenyl)-3-(1-methyl-1H-pyrrol-3-yl)-5-oxo-1,5-dihydropyrazol-4-ylidene]hydrazino}-2-hydroxy-3′-tetrazol-5-ylbiphenyl;
3′-{N′-[1-(3,4-dimethylphenyl)-3-furan-2-yl-5-oxo-1,5-dihydropyrazol-4-ylidene]hydrazino}-2′-hydroxybiphenyl-3-carboxylic acid;
3-{N′-[1-(3,4-dimethylphenyl)-3-furan-2-yl-5-oxo-1,5-dihydropyrazol-4-ylidene]hydrazino}-2-hydroxy-3′-tetrazol-5-ylbiphenyl;
N-(2′-hydroxy-3′-{N′-[3-methyl-5-oxo-1-(4-trifluoromethyl-phenyl)-1,5-dihydro-pyrazol-4-ylidene]hydrazino}biphenyl-3-yl)-1,1,1-trifluoromethanesulfonamide;
N-(2′-hydroxy-3′-{N′-[1-(3-fluoro-4-methylphenyl)-3-methyl-5-oxo-1,5-dihydro-pyrazol-4-ylidene]hydrazino}biphenyl-3-yl)-1,1,1-trifluoromethanesulfonamide;
N-(2′-hydroxy-3′-{N′-[1-(4-fluoro-3-methylphenyl)-3-methyl-5-oxo-1,5-dihydro-pyrazol-4-ylidene]hydrazino}biphenyl-3-yl)-1,1,1-trifluoromethanesulfonamide;
N-(2′-hydroxy-3′-{N′-[1-(3,4-difluorophenyl)-3-methyl-5-oxo-1,5-dihydro-pyrazol-4-ylidene]hydrazino}biphenyl-3-yl)-1,1,1-trifluoromethanesulfonamide;
N-(3′-{N′-[1-(3,4-dimethylphenyl)-3-methyl-5-oxo-1,5-dihydropyrazol-4-ylidene]hydrazino}-2′-hydroxybiphenyl-3-yl)guanidine;
3′-{N′-[1-(3,4-dimethylphenyl)-3-ethyl-5-oxo-1,5-dihydropyrazol-4-ylidene]hydrazino}-2′-hydroxybiphenyl-3-carboxylic acid;
3-{N′-[1-(3,4-dimethylphenyl)-3-ethyl-5-oxo-1,5-dihydropyrazol-4-ylidene]hydrazino}-2-hydroxy-3′-tetrazol-5-ylbiphenyl;
3′-{N′-[1-(3,4-dimethylphenyl)-5-oxo-3-thien-2-yl-1,5-dihydropyrazol-4-ylidene]hydrazino}-2′-hydroxybiphenyl-3-carboxylic acid;
3′-{N′-[3-cyclopropyl-1-(3,4-dimethylphenyl)-5-oxo-1,5-dihydropyrazol-4-ylidene]hydrazino}-2′-hydroxybiphenyl-3-carboxylic acid;
3′-{N′-[1-(3,4-dimethylphenyl)-5-oxo-3-thiazol-2-yl-1,5-dihydropyrazol-4-ylidene]hydrazino}-2′-hydroxybiphenyl-3-carboxylic acid;
3′-{N′-[1-(3,4-dimethylphenyl)-5-oxo-1,5-dihydropyrazol-4-ylidene]hydrazino}-2′-hydroxybiphenyl-3-carboxylic acid;
3′-{N′-[1-(3,4-dimethylphenyl)-3-(1-methylethyl)-5-oxo-1,5-dihydropyrazol-4-ylidene]hydrazino}-2′-hydroxybiphenyl-3-carboxylic acid;
3′-{N′-[3-(benzyloxymethyl)-1-(3,4-dimethylphenyl)-5-oxo-1,5-dihydropyrazol-4-ylidene]hydrazino}-2′-hydroxybiphenyl-3-carboxylic acid;
3′-{N′-[3-ethyl-5-oxo-1-(4-trifluoromethylphenyl)-1,5-dihydropyrazol-4-ylidene]hydrazino}-2′-hydroxybiphenyl-3-carboxylic acid;
3′-{N′-[5-oxo-1-(4-trifluoromethylphenyl)-1,5-dihydropyrazol-4-ylidene]hydrazino}-2′-hydroxybiphenyl-3-carboxylic acid;
3′-{N′-[-1-(3,4-dimethylphenyl)-3-hydroxymethyl-5-oxo-1,5-dihydropyrazol-4-ylidene]hydrazino}-2′-hydroxybiphenyl-3-carboxylic acid;
3′-{N′-[3-benzyloxymethyl-5-oxo-1-(4-trifluoromethylphenyl)-1,5-dihydropyrazol-4-ylidene]hydrazino}-2′-hydroxybiphenyl-3-carboxylic acid;
3′-{N′-[-1-(3,4-dimethylphenyl)-3-methylsulfanylmethyl-5-oxo-1,5-dihydropyrazol-4-ylidene]hydrazino}-2′-hydroxybiphenyl-3-carboxylic acid;
3′-{N′-[-1-(3,4-dimethylphenyl)-5-oxo-3-thiophen-3-yl-1,5-dihydropyrazol-4-ylidene]hydrazino}-2′-hydroxybiphenyl-3-carboxylic acid;
3′-{N′-[5-oxo-1-(4-trifluoromethylphenyl)-3-thiophen-3-yl-1,5-dihydropyrazol-4-ylidene]hydrazino}-2′-hydroxybiphenyl-3-carboxylic acid;
3′-{N′-[5-oxo-1-(4-trifluoromethylphenyl)-3-methylsulfanylmethyl-1,5-dihydropyrazol-4-ylidene]hydrazino}-2′-hydroxybiphenyl-3-carboxylic acid;
N-(3′-{N′-[1-(3,4-dimethylphenyl)-3-methyl-5-oxo-1,5-dihydro-pyrazol-4-ylidene]hydrazino}-2′-hydroxybiphenyl-3-yl)methanesulfonamide;
3′-{N′-[1-benzo[1,3]dioxol-5-yl-3-methyl-5-oxo-1,5-dihydropyrazol-4-ylidene]hydrazino}-2′-hydroxybiphenyl-3-carboxylic acid;
3′-{N′-[1-(3,5-dimethylphenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-4′-hydroxybiphenyl-4-carboxylic acid;
3′-{N′-[1-(3-chloro-4-methylphenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-4′-hydroxybiphenyl-3-carboxylic acid;
3′-{N′-[1-(3,4-dimethylphenyl)-3-methyl-5-oxo-1,5-dihydropyrazol-4-ylidene]hydrazino}-2′-hydroxybiphenyl-3-phosphonic acid;
3′-{N′-[1-(3,4-dimethylphenyl)-3-methyl-5-oxo-1,5-dihydropyrazol-4-ylidene]hydrazino}-2′-hydroxybiphenyl-3,4-dicarboxylic acid;
2′,6-dihydroxy-3′-{N′-[1-(3,4-dimethylphenyl)-3-methyl-5-oxo-1,5-dihydropyrazol-4-ylidene]hydrazino}biphenyl-3-carboxylic acid;
4-aza-3′-{N′-[1-(3,4-dimethylphenyl)-3-methyl-5-oxo-1,5-dihydropyrazol-4-ylidene]hydrazino}-2′-hydroxybiphenyl-5-carboxylic acid;
3′-{N′-[1-(3,4-dimethylphenyl)-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-2′-hydroxybiphenyl-3-sulfonic acid;
5-(3′-{N′-[1-(3,4-Dimethylphenyl)-3-methyl-5-oxo-1,5-dihydropyrazol-4-ylidene]hydrazino}-2′-hydroxybiphenyl-3-ylmethylene)thiazolidine-2,4-dione; and

pharmaceutically acceptable salts, hydrates, solvates and esters thereof.

In one embodiment the hydroxyl-1-azo-benzene derivative TPO mimetic is (Z)-3′-(2-(1-(3,4-dimethylphenyl)-3-methyl-5-oxo-1H-pyrazol-4(5H)-ylidene)hydrazinyl)-2′-hydroxybiphenyl-3-carboxylic acid, i.e., Eltrombopag, having the following chemical structure:

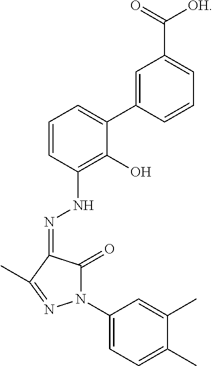

Alternatively, the TPO mimetic of the present invention comprises Eltrombopag ethanolamine salt or other Eltrombopag polymorphs as described in U.S. Pat. No. 8,217,021 to Leksic et al., which is hereby incorporated by reference in its entirety.

In another embodiment of the present invention, the non-peptide TPO mimetic is 5-[(2-{1-[5-(3,4-dichlorophenyl)-4-hydroxy-3-thienyl]ethylidene}hydrazino)carbonyl]-2-thiophenecarboxylic acid (NIP-004), a pharmaceutically acceptable salt, a hydrate, a solvate, an ester, or a polymorph thereof as described by Nakamura et al., “A Novel Nonpeptidyl Human c-Mpl Activator Stimulates Human Megakaryopoiesis and Thrombopoiesis,” Blood 107(11) 4300-4307 (2006), which is hereby incorporated by reference in its entirety.

In another embodiment the non-peptide TPO mimetic comprises AKR-501 (YM477) as described by Fukushima-Shintani et al., “AKR-501 (YM477) A Novel Orally-Active Thrombopoietin Receptor Agonist,” Eur. J. Haematol. 82(4):247-54 (2009), which is hereby incorporated by reference in its entirety.

In another embodiment, the non-peptide TPO mimetic is a small molecule having the formula of Formula VII as disclosed in U.S. Pat. No. 7,314,887 to Chen et al., which is hereby incorporated by reference in its entirety.

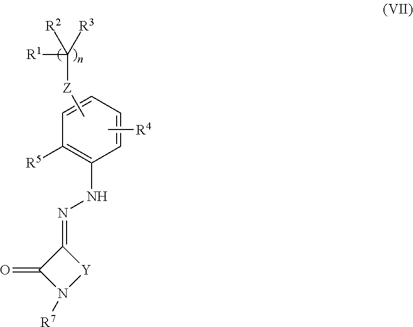

or a pharmaceutically acceptable salt, ester, amide, or prodrug thereof, wherein:
R¹ is selected from CO₂R¹⁰, CONR¹⁰R¹¹, SO₃R¹⁰, and a carboxylic acid bioisostere;
R² and R³ are each independently selected from null, hydrogen, OR¹², NR¹²R¹³, an optionally substituted C₁-C₄ aliphatic, an optionally substituted C₁-C₄ haloaliphatic, an optionally substituted C₁-C₄ heteroaliphatic, an optionally substituted ring, and (CH₂)ₘR¹⁴; or R² and R³ taken together form an optionally substituted olefin; or R² and R³ are linked to form an optionally substituted C₃-C₈ ring;
R⁴ is selected from hydrogen, F, Cl, Br, C₁-C₄ aliphatic, C₁-C₄ haloaliphatic, C₁-C₄ heteroaliphatic, and a ring;
R⁵ is selected from hydrogen, OR¹⁰, SR¹⁰, NHR¹¹, and CO₂H;
R⁶ is selected from hydrogen, OR¹², NR¹²R¹³, F, Cl, Br, C₁-C₄ alkyl, C₁-C₄ haloalkyl, C₁-C₄ heteroalkyl, and a ring;
R⁷ is selected from hydrogen, an optionally substituted C₁-C₈ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴;
R¹⁰ is selected from hydrogen, an optionally substituted C₁-C₄ aliphatic, C₁-C₄ haloaliphatic, C₁-C₄ heteroaliphatic, and a ring;
R¹¹ is selected from hydrogen, SO₂R¹⁵, C₁-C₄ aliphatic, C₁-C₄ haloaliphatic, C₁-C₄ heteroaliphatic, and a ring;
R¹² and R¹³ are each independently selected from hydrogen, an optionally substituted C₁-C₄ aliphatic, an optionally substituted C₁-C₄ haloaliphatic, an optionally substituted C₁-C₄ heteroaliphatic, an optionally substituted ring, and (CH₂)ₘR¹⁴; or one of R¹² and R¹³ is an optionally substituted C₂-C₆ aliphatic or an optionally substituted ring and the other of R¹² and R¹³ is null; or R¹² and R¹³ are linked to form an optionally substituted C₃-C₈ ring;
R¹⁴ is selected from an optionally substituted aryl and an optionally substituted heteroaryl;
R¹⁵ is selected from hydrogen, C₁-C₃ aliphatic, C₁-C₃ haloaliphatic, and a ring;
Y is a 1-4 atom spacer comprising one or more groups selected from an optionally substituted C₁-C₆ aliphatic, an optionally substituted C₁-C₆heteroaliphatic, an optionally substituted phenyl, an optionally substituted heteroaryl, an optionally substituted C₃-C₅ heterocycle, and an optionally substituted alicyclic, provided that Y is not —N═CR⁶— orientated to form a dihydropyrazole;
Z is selected from:
   a 2-5 atom spacer selected from an optionally substituted C₆-C₁₀ aryl and an optionally substituted C₁-C₈ heteroaryl, and
   a 1-5 atom spacer of selected from an optionally substituted C₁-C₆ aliphatic, an optionally substituted C₁-C₆heteroaliphatic, and an optionally substituted C₁-C₆ haloaliphatic;
m is 0, 1, or 2; and
n is 0 or 1.

In another embodiment, the non-peptide TPO mimetic comprises a small molecule having Formula VIII as disclosed in U.S. Pat. No. 7,314,887 to Chen et al., which is hereby incorporated by reference in its entirety.

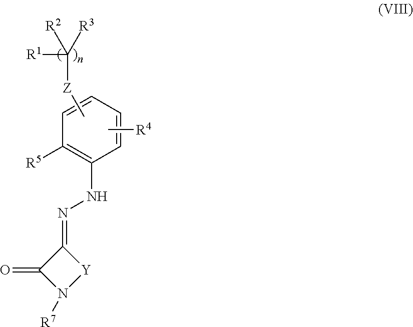

or a pharmaceutically acceptable salt, ester, amide, or prodrug thereof, wherein:
R¹ is selected from CO₂R¹⁰, CONR¹⁰R¹¹, SO₃R¹⁰, and a carboxylic acid bioisostere;
R² and R³ are each independently selected from null, hydrogen, OR¹², NR¹²R¹³, an optionally substituted C₁-C₄ aliphatic, an optionally substituted C₁-C₄ haloaliphatic, an optionally substituted C₁-C₄ heteroaliphatic, an optionally substituted ring, and (CH₂)ₘR¹⁴; or
   R² and R³ taken together form an optionally substituted olefin; or R² and R³ are linked to form an optionally substituted C₃-C₈ ring;
R⁴ is selected from hydrogen, F, Cl, Br, C₁-C₄ aliphatic, C₁-C₄ haloaliphatic, C₁-C₄ heteroaliphatic, and a ring;
R⁵ is selected from hydrogen, OR¹⁰, SR¹⁰, NHR¹¹, and CO₂H;
R⁶ is selected from hydrogen, OR¹², NR¹²R¹³, F, Cl, Br, C₁-C₄ alkyl, C₁-C₄ haloalkyl, C₁-C₄ heteroalkyl, and a ring;
R⁷ is selected from hydrogen, an optionally substituted C₁-C₈ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴;
R⁸ and R⁹ are each independently selected from hydrogen, F, Cl, Br, CO₂R¹⁰, NO₂, CN, SO₂R¹⁰, (CH₂)ₘR¹⁴, C₁-C₄ aliphatic, C₁-C₄ haloaliphatic, C₁-C₄ heteroaliphatic, C₁-C₄ heterohaloaliphatic, and a ring;
R¹⁰ is selected from hydrogen, an optionally substituted C₁-C₄ aliphatic, C₁-C₄ haloaliphatic, C₁-C₄ heteroaliphatic, and a ring;
R¹¹ is selected from hydrogen, SO₂R¹⁵, C₁-C₄ aliphatic, C₁-C₄ haloaliphatic, C₁-C₄ heteroaliphatic, and a ring;
R¹² and R¹³ are each independently selected from hydrogen, an optionally substituted C₁-C₄ aliphatic, an optionally substituted C₁-C₄ haloaliphatic, an optionally substituted C₁-C₄ heteroaliphatic, an optionally substituted ring, and (CH₂)ₘR¹⁴; or one of R¹² and R¹³ is an optionally substituted C₂-C₆ aliphatic or an optionally substituted ring and the other of R¹² and R¹³ is null; or R¹² and R¹³ are linked to form an optionally substituted C₃-C₈ ring;
R¹⁴ is selected from an optionally substituted aryl and an optionally substituted heteroaryl;
R¹⁵ is selected from hydrogen, C₁-C₃ aliphatic, C₁-C₃ haloaliphatic, and a ring;
Q is selected from O and S;
X is selected from O, S, NR¹⁰, and CR¹⁰R¹¹;
Y is selected from

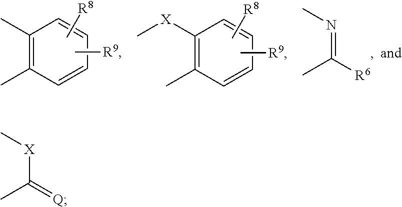

Z is selected from:
   a 2-5 atom spacer selected from an optionally substituted C₆-C₁₀ aryl and an optionally substituted C₁-C₈ heteroaryl, and
   a 1-5 atom spacer of selected from an optionally substituted C₁-C₆ aliphatic, an optionally substituted C₁-C₆ heteroaliphatic, and an optionally substituted C₁-C₆ haloaliphatic;
m is 0, 1, or 2; and
n is 0 or 1.

In another embodiment, the non-peptide TPO mimetic is a small molecule having Formula IX as disclosed in U.S. Pat. No. 7,314,887 to Chen et al., which is hereby incorporated by reference in its entirety.

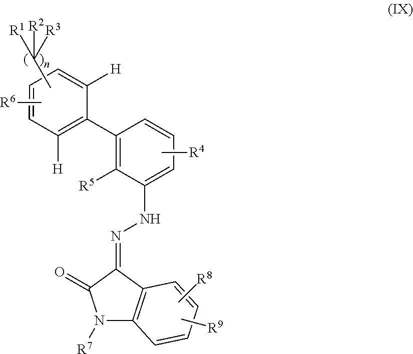

or a pharmaceutically acceptable salt, ester, amide, or prodrug thereof, wherein:
R¹ is selected from CO₂R¹⁰, CONR¹⁰R¹¹, SO₃R¹⁰, and a carboxylic acid bioisostere;
R² and R³ are each independently selected from null, hydrogen, OR¹², NR¹²R¹³, an optionally substituted C₁-C₄ aliphatic, an optionally substituted C₁-C₄ haloaliphatic, an optionally substituted C₁-C₄ heteroaliphatic, an optionally substituted ring, and (CH₂)ₘR¹⁴; or R² and R³ taken together form an optionally substituted olefin; or R² and R³ are linked to form an optionally substituted C₃-C₈ ring;
R⁴ is selected from hydrogen, F, Cl, Br, C₁-C₄ aliphatic, C₁-C₄ haloaliphatic, C₁-C₄ heteroaliphatic, and a ring;
R⁵ is selected from hydrogen, OR¹⁰, SR¹⁰, NHR¹¹, and CO₂H;

R⁶ is selected from hydrogen, OR¹², NR¹²R¹³, F, Cl, Br, C₁-C₄ alkyl, C₁-C₄haloalkyl, and C₁-C₄ heteroalkyl;
R⁷ is selected from hydrogen, an optionally substituted C₁-C₈ aliphatic, an optionally substituted C₁-C₈ haloaliphatic, an optionally substituted C₁-C₈ heteroaliphatic, an optionally substituted C₁-C₈ heterohaloaliphatic, an optionally substituted ring, and (CH₂)ₘR¹⁴;
R⁸ and R⁹ are each independently selected from hydrogen, F, Cl, Br, CO₂R¹⁰, NO₂, CN, SO₂R¹⁰, (CH₂)ₘR¹⁴, C₁-C₄ aliphatic, C₁-C₄ haloaliphatic, C₁-C₄ heteroaliphatic, and C₁-C₄ heterohaloaliphatic;
R¹⁰ is selected from hydrogen, an optionally substituted C₁-C₄ aliphatic, C₁-C₄ haloaliphatic, C₁-C₄ heteroaliphatic, and a ring;
R¹¹ is selected from hydrogen, SO₂R¹⁵, C₁-C₄ aliphatic, C₁-C₄ haloaliphatic, C₁-C₄ heteroaliphatic, and a ring;
R¹² and R¹³ are each independently selected from hydrogen, an optionally substituted C₁-C₄ aliphatic, an optionally substituted C₁-C₄ haloaliphatic, an optionally substituted C₁-C₄ heteroaliphatic, an optionally substituted ring, and (CH₂)ₘR¹⁴; or one of R¹² and R¹³ is an optionally substituted C₂-C₆ aliphatic or an optionally substituted ring and the other of R¹² and R¹³ is null; or R¹² and R¹³ are linked to form an optionally substituted C₃-C₈ ring;
R¹⁴ is selected from an optionally substituted aryl and an optionally substituted heteroaryl;
R¹⁵ is selected from hydrogen, C₁-C₃ aliphatic, C₁-C₃ haloaliphatic, and a ring;
m is 0, 1, or 2; and
n is 0 or 1.

In another embodiment of the present invention, the c-Mpl agonist comprises a TPO peptide mimetic or peptibody. Suitable peptide mimetics and peptibodies are disclosed in U.S. Patent Application Publication No. 20090011497 to Hosung et al., which is hereby incorporated by reference.

Briefly, suitable peptide thrombopoietin mimetic peptides (TMPs) comprises the sequence of SEQ ID NO: 25 as follows:

X¹—X²—X³—X⁴-G-P-T-L-X⁹—X¹⁰—W-L-X¹³—X¹⁴—X¹⁵—X¹⁶—X¹⁷—X¹⁸
wherein X¹—X⁴, X⁹, X¹⁰, and X¹³—X¹⁸ are each independently an amino acid. Preferred amino acid residues of the above sequence are defined in Table 1 below.

TABLE 1
Preferred Amino Acid Residues for SEQ ID NO: 25

| Position | Amino Acid Residue |
|---|---|
| X¹ | A, V, W, M, G, Y, C, Q, E, R, H |
| X² | A, V, L, I, G, S, C |
| X³ | L, I, P, W, G, S, D, K, R |
| X⁴ | L, G, Q, D, E, H |
| X⁹ | K, R |
| X¹⁰ | Q, E |
| X¹³ | A, V, L, S, Q, E, R |
| X¹⁴ | A, W, T, Y, C, Q |
| X¹⁵ | V, L, G, Y, R |
| X¹⁶ | A, L, F, G, R |
| X¹⁷ | A, V, L, M, G, C, Q, N |
| X¹⁸ | A, V, P, M, F, G, C, Q, K |

Preferred TMP sequences of the present invention are identified as TMP2-TMP29 in Table 2 of U.S. Patent Application Publication No. 20090011497 to Hosung et al., which is hereby incorporated by reference in its entirety.

In addition to the TPO mimetic peptides described above, peptide compounds wherein one or more of the above TMPs encompassed by SEQ ID NO: 25 are attached or otherwise linked to each other, to a linker (LN), and/or to a vehicle (V). TPO mimetics may be linked in tandem (i.e., sequentially, N-terminus to C-terminus) or in parallel (i.e., N- to N-terminus or C- to C-terminus). TMPs may be attached to other TMPs or the same TMP, with or without linkers. TMPs may also be attached to other TMPs or the same TMP with or without linkers and with or without vehicles. Peptide-linker-vehicle compounds of the present invention may be described by the following formula:

(V1)ᵥ-(LN1)₁-(TMP1)ₐ-(LN2)ₘ-(TMP2)_{b}-(LN3)ₙ-(TMP3)_{c}-(LN4)ₒ-(TMP4)_{d}-(V2)_{w}

wherein:

V1 and V2 are vehicles; LN1, LN2, LN3 and LN4 are each independently linkers; TMP1, TMP2, TMP3 and TMP4 are each independently peptide sequences of SEQ ID NO: 25; a, b, c and d and l, m, n and o are each independently an integer from zero to twenty, and v and w are each independently an integer from zero to one.

Exemplary compounds of this embodiment are represented by formulae:

TMP1-V1

TMP1-LN1-V1

TMP1-TMP2-V1

TMP1-LN1-TMP2-LN2-V1

and additional multimers thereof wherein V1 is a vehicle (preferably an Fc domain) and is attached at the C-terminus of a TMP, either with or without a linker;

V1-TMP1

V1-LN1-TMP1

V1-TMP1-TMP2

V1-LN1-TMP1-LN2-TMP2

and multimers thereof wherein V1 is a vehicle (preferably an Fc domain) and is attached at the N-terminus of a TMP, either with or without a linker.

In another embodiment, the one or more TMPs is covalently bonded or otherwise linked or attached to another TMP peptide via a “linker” group (LN1, LN2, etc.). Any linker group is optional. When it is present, it is not critical what its chemical structure, since it serves primarily as a spacer. The linker should be chosen so as not to interfere with the biological activity of the final compound and also so that immunogenicity of the final compound is not significantly increased. The linker is preferably made up of amino acids linked together by peptide bonds. Thus, in preferred embodiments, the linker is made up of from 1 to 30 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly)₄, (Gly)₅), poly(Gly-Ala), and polyalanines.

Non-peptide linkers are also possible. For example, alkyl linkers such as —NH—(CH₂)ₙ—C(O)—, wherein n=2-20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker,

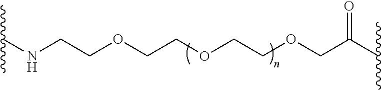

wherein n is such that the linker has a molecular weight of 100 to 5000 kD, preferably 100 to 500 kD. The peptide linkers may be altered to form derivatives in the same manner as described above.

In general a linker of a length of about 0-14 sub-units (e.g., amino acids) is preferred for the TMPs described herein. The peptide linkers may be altered to form derivatives in the same manner as described above for the TMPs. In addition, the TMP compounds of this embodiment may further be linear or cyclic. By “cyclic” is meant that at least two separated, i.e., non-contiguous, portions of the molecule are linked to each other. For example, the amino and carboxy terminus of the ends of the molecule could be covalently linked to form a cyclic molecule. Alternatively, the molecule could contain two or more Cys residues (e.g., in the linker), which could cyclize via disulfide bond formation. It is further contemplated that more than one tandem peptide dimer can link to form a dimer of dimers. Thus, for example, a tandem dimer containing a Cys residue can form an intermolecular disulfide bond with a Cys of another such dimer. Thus, in preferred embodiments, the linker comprises (LN1)ₙ, wherein LN1 is a naturally occurring amino acid or a stereoisomer thereof and “n” is any one of 1 through 20.

Further preferred peptide-linker molecules include:

i) TMP1-LN1-TMP2-LN2

ii) LN1-TMP1-LN2-TMP2

iii) LN1-TMP1-LN2-TMP1

iv) TMP1-LN1-TMP1-LN1-TMP1-LN1

v) LN1-TMP1-LN2-TMP2-LN3-TMP3-LN4-TMP4

wherein LN1-LN4 are each independent linkers.

In yet another embodiment, peptides or peptide compounds of the present invention may be linked or attached to a vehicle (V). A vehicle generally refers to a molecule that prevents degradation and/or increases half-life, reduces toxicity, reduces immunogenicity, or increases biological activity of a therapeutic protein. The vehicle (V) may be attached to a peptide through the N-terminus, C terminus, peptide backbone or a sidechain.

The vehicle (V) may be a carrier molecule, such as a linear polymer (e.g., polyethylene glycol, polylysine, dextran, etc.), a branched-chain polymer (see, for example, U.S. Pat. No. 4,289,872 to Denkenwalter et al., U.S. Pat. No. 5,229,490 to Tam; WO 93/21259 by Frechet et al., which are hereby incorporated by reference in their entirety); a lipid; a cholesterol group (such as a steroid); or a carbohydrate or oligosaccharide. Other possible carriers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337, which are hereby incorporated by reference in their entirety. Still other useful polymers known in the art include monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers. Exemplary vehicles also include: an Fc domain; other proteins, polypeptides, or peptides capable of binding to a salvage receptor; human serum albumin (HSA); a leucine zipper (LZ) domain; polyethylene glycol (PEG), including 5 kD, 20 kD, and 30 kD PEG, as well as other polymers; dextran; and other molecules known in the art to provide extended half-life and/or protection from proteolytic degradation or clearance.

An exemplary carrier is polyethylene glycol (PEG). The PEG group may be of any convenient molecular weight and may be straight chain or branched. The average molecular weight of the PEG will preferably range from about 2 kDa to about 100 kDa, more preferably from about 5 kDa to about 50 kDa, most preferably from about 5 kDa to about 10 kDa.

The PEG groups will generally be attached to the compounds of the invention via acylation, reductive alkylation, Michael addition, thiol alkylation or other chemoselective conjugation/ligation methods through a reactive group on the PEG moiety (e.g., an aldehyde, amino, ester, thiol, -haloacetyl, maleimido or hydrazino group) to a reactive group on the target compound (e.g., an aldehyde, amino, ester, thiol, haloacetyl, maleimido or hydrazino group).

An exemplary pegylated TPO mimetic is Peg-TPOmp as described by Cerneus et al., “Stimulation of Platelet Production in Healthy Volunteers by a Novel Pegylated Peptide-Based Thrombopoietin (TPO) Receptor Agonist,” Blood 106: (2005); and Kuter, “New Thrombopoietic Growth Factors,” Blood 109(11):4607-4616 (2007), which are hereby incorporated by reference in their entirety.

In another embodiment of the present invention, the vehicle (V) may comprise one or more antibody Fc domains. Thus, the peptide compounds described above may further be fused to one or more Fc domains, either directly or through linkers. Such compounds are referred to as peptibodies. The Fc vehicle may be selected from the human immunoglobulin IgG-1 heavy chain (see Ellison et al., Nucleic Acids Res. 10:4071-4079 (1982), which is hereby incorporate by reference in its entirety) or any other Fc sequence known in the art (e.g., other IgG classes including but not limited to IgG-2, IgG-3 and IgG-4, or other immunoglobulins).

It is well known that Fc regions of antibodies are made up of monomeric polypeptide segments that may be linked into dimeric or multimeric forms by disulfide bonds or by non-covalent association. The number of intermolecular disulfide bonds between monomeric subunits of native Fc molecules ranges from 1 to 4 depending on the class (e.g., IgG, IgA, IgE) or subclass (e.g., IgG1, IgG2, IgG3, IgA1, IgGA2) of antibody involved. The term “Fc” as used herein is generic to the monomeric, dimeric, and multimeric forms of Fc molecules. It should be noted that Fc monomers will spontaneously dimerize when the appropriate Cys residues are present unless particular conditions are present that prevent dimerization through disulfide bond formation. Even if the Cys residues that normally form disulfide bonds in the Fc dimer are removed or replaced by other residues, the monomeric chains will generally dimerize through non-covalent interactions. The term “Fc” herein is used to mean any of these forms: the native monomer, the native dimer (disulfide bond linked), modified dimers (disulfide and/or non-covalently linked), and modified monomers (i.e., derivatives).

Variants, analogs or derivatives of the Fc portion may be constructed by, for example, making various substitutions of residues or sequences. Variant (or analog) polypeptides include insertion variants, wherein one or more amino acid residues supplement an Fc amino acid sequence. Insertions may be located at either or both termini of the protein, or may be positioned within internal regions of the Fc amino acid sequence. Insertional variants with additional residues at either or both termini can include for example, fusion proteins and proteins including amino acid tags or labels. For example, the Fc molecule may optionally contain an N-terminal Met, especially when the molecule is expressed recombinantly in a bacterial cell such as E. coli.

In Fc deletion variants, one or more amino acid residues in an Fc polypeptide are removed. Deletions can be effected at one or both termini of the Fc polypeptide, or with removal of one or more residues within the Fc amino acid sequence. Deletion variants, therefore, include all fragments of an Fc polypeptide sequence.

In Fc substitution variants, one or more amino acid residues of an Fc polypeptide are removed and replaced with alternative residues. In one aspect, the substitutions are conservative in nature, however, the invention embraces substitutions that are also non-conservative.

For example, cysteine residues can be deleted or replaced with other amino acids to prevent formation of some or all disulfide crosslinks of the Fc sequences. One may remove each of these cysteine residues or substitute one or more such cysteine residues with other amino acids, such as Ala or Ser. As another example, modifications may also be made to introduce amino acid substitutions to (1) ablate the Fc receptor binding site; (2) ablate the complement (Clq) binding site; and/or to (3) ablate the antibody dependent cell-mediated cytotoxicity (ADCC) site. Such sites are known in the art, and any known substitutions are within the scope of Fc as used herein.

Likewise, one or more tyrosine residues can be replaced by phenylalanine residues as well. In addition, other variant amino acid insertions, deletions (e.g., from 1-25 amino acids) and/or substitutions are also contemplated and are within the scope of the present invention. Conservative amino acid substitutions will generally be preferred. Furthermore, alterations may be in the form of altered amino acids, such as peptidomimetics or D-amino acids.

Fc sequences of the present invention may also be derivatized, i.e., bearing modifications other than insertion, deletion, or substitution of amino acid residues. Preferably, the modifications are covalent in nature, and include for example, chemical bonding with polymers, lipids, other organic, and inorganic moieties. Derivatives of the invention may be prepared to increase circulating half-life, or may be designed to improve targeting capacity for the polypeptide to desired cells, tissues, or organs.

It is also possible to use the salvage receptor binding domain of the intact Fc molecule as the Fc part of the inventive compounds, such as described in WO 96/32478; WO 97/34631, each of which is hereby incorporated by reference in its entirety.

The Fc fusions may be at the N- or C-terminus of TMP₁ or TMP₂ or at both the N- and C-termini of TMP₁ or TMP₂. Similarly, the Fc fusions may be at the N- or C-terminus of the Fc domain.

Preferred compounds of the present invention include IgG1 Fc fusion dimers linked or otherwise attached to dimers or multimers of the TMPs disclosed herein. In such cases, each Fc domain will be linked to a dimer or multimer of TMP peptides, either with or without linkers.

An exemplary TMP peptibody comprises AMG 531 (also known as Romiplostim and Nplate). AMG 531 is a peptide TPO mimetic composed of an IgG Fc fragment to which are attached four 14-amino acid TMPs that activate c-Mpl receptor by binding to the extracytoplasmic domain just like endogenous TPO (Kutter D J, “Biology and Chemistry of Thrombopoietic Agents,” Semin Hematol. 47(3):243-8 (2010), which is hereby incorporated by reference in its entirety).

Multiple vehicles may also be used; e.g., Fc's at each terminus or an Fc at a terminus and a PEG group at the other terminus or a sidechain.

Exemplary peptide-vehicle compounds are provided in Table 4 of U.S. Patent Application Publication No. 20090011497 to Hosung et al., which is hereby incorporated by reference in its entirety.

Other suitable TPO peptide mimetics and peptibodies are disclosed in U.S. Patent Application Publication No. 2011/0071077 to Nichol et al., which is hereby incorporated by reference in its entirety.

In another aspect of the present invention, the c-Mpl receptor agonist is an agonist antibody. A suitable agonist antibody, is an antibody that activates a thrombopoietin receptor, which preferably comprises a mammalian c-mpl, more preferably human c-mpl. Usually the antibody will be a full length antibody such as an IgG antibody. Suitable representative fragment agonist antibodies include Fv, ScFv, Fab, F(ab′)₂ fragments, as well as diabodies and linear antibodies. These fragments may be fused to other sequences including, for example, the F″ or Fc region of an antibody, a “leucine zipper” or other sequences including pegylated sequences or Fc mutants used to improve or modulate half-life. Normally the antibody is a human antibody and may be a non-naturally occurring antibody, including affinity matured antibodies.

Suitable c-Mpl agonist antibodies are disclosed in U.S. Pat. No. 6,342,220 to Adams et al., which is hereby incorporated by reference in its entirety. Representative antibodies that activate c-mpl are selected from the group 12E10, 12B5, 10F6 and 12D5, and affinity matured derivatives thereof. The amino acid sequences of the 12E10 antibody, the 12B5 antibody, the 10F6 antibody, and the 12D5 antibody are identified by Sequence Identifiers 31-34 of U.S. Pat. No. 6,342,220 to Adams et al., which is hereby incorporated by reference in its entirety.

Suitable c-Mpl agonist antibodies also include TPO minibodies, such as VB228 sc(Fv)₂ (Orita et al., “A Novel Therapeutic Approach for Thrombocytopenia by Minibody Agonist of the Thrombopoietin Receptor,” Blood 105:562-66 (2005), which is hereby incorporated by reference in its entirety). Other suitable c-Mpl agonist antibodies are described in Kai et al., “Domain Subclass Conversion Improved Activity of Anti-Mpl Agonist Antibodies in the Form of Whole IgG,” Blood 108 (2006), which is hereby incorporated by reference in its entirety.

### EXAMPLES

The following examples are provided to illustrate embodiments of the present invention but are by no means intended to limit its scope.

### Example 1

### Generation of Human TPO Receptor (c-Mpl) cDNA Knock-in Mouse/Mpl^{hmMPL}

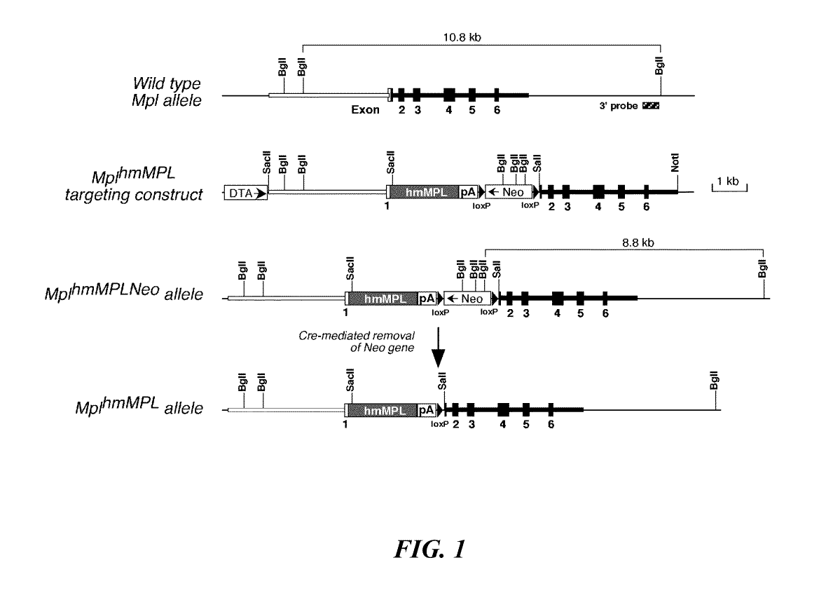

To generate human c-mplc DNA knock-in mice, mouse 129S6 BAC genomic DNA was obtained from The BACPAC Resource Center (BPRC) at the Children's Hospital Oakland Research Institute in Oakland, Calif., USA. The Mpl^{hmMPLNeo} knock-in construct was generated by inserting 3.5 kb c-mpl 5′ flanking sequence ending at the 20th nucleotide upstream of the translation initiation codon ATG and 4.0 kb 3′ sequence starting from the 18th nucleotide upstream of ATG into 5′ and 3′ multiple cloning sites of pKIIlox vector at the SacII-XhoI and SalI-NotI sites, respectively. The SalI-SalI human-mouse hybrid cDNA fragment, which contains human mpl extracellular and transmembrane domains (amino acids 1-513, NCBI Accession No. NM_{—}005373), mouse mpl cytoplasmic domain (amino acids 513-633, NCBI Accession No. NM_{—}001122949), and a SV40 polyadenylation sequences, was inserted at an XhoI site at the 3′ end of the 5′ flanking sequences (FIG. 1). To generate Mpl^{hmMPLNeo}-null mice, NotI-linearized Mpl^{hmMPL} targeting construct was electroporated into W4 mouse embryonic stem (ES) cells. Positive targeted ES cells were obtained by drug selection with G418 and confirmed by Southern blotting genotyping. Correctly targeted ES clones were injected into C57BL/6J blastocysts to generate mouse chimeras. Mpl^{hmMPLNeo/+} heterozygous mice were generated by breeding Mpl^{hmMPLNeo/+} chimeras with wild type C57BL/6J mice. Crossing of Mpl^{hmMPLNeo/+} and Tg(CMV-cre)1Cgn/J (Jackson Laboratory) mice resulted in the removal of neomycin resistant gene cassette to create Mpl^{hmMPL/+} mice.

### Example 2

### Experimental Verification of Human TPO Receptor (c-Mpl) cDNA Knock-in Mouse Mpl^{hmMPL} at the DNA Level (Genome Typing)

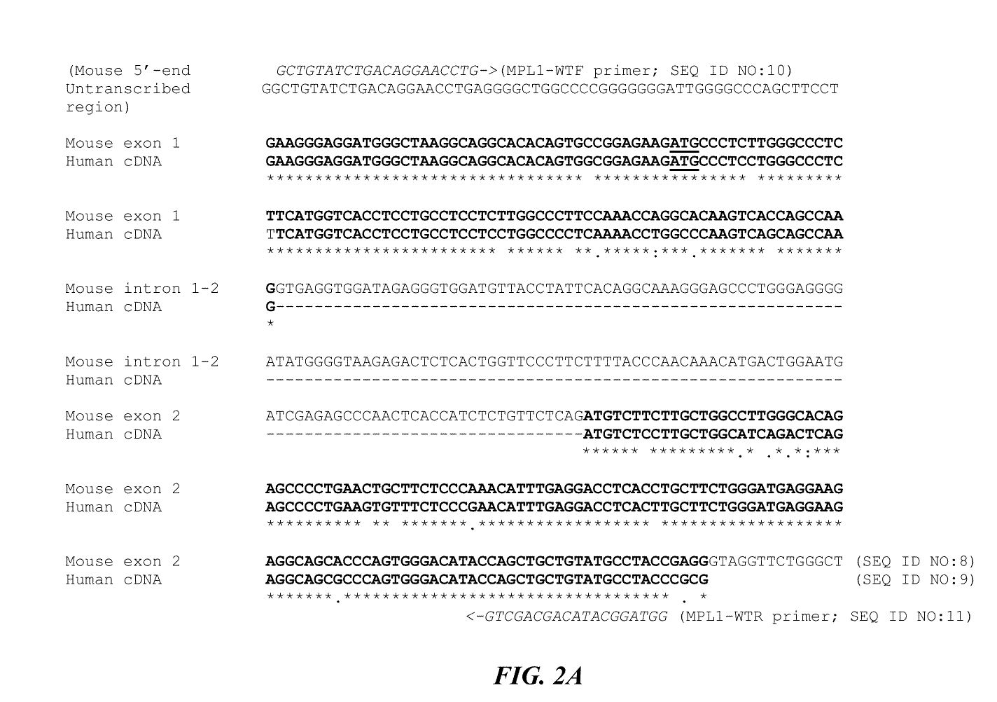

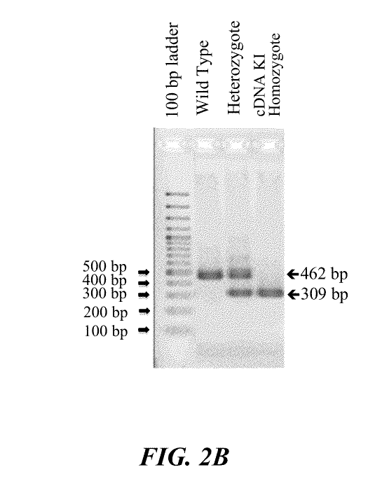

To experimentally verify that the human c-mpl cDNA sequence encoding the extracellular and trans-membrane domains was successfully knocked into the mouse genome, genomic DNA PCR was carried out using a mouse-specific forward primer and a common reverse primer. As shown in FIG. 2A, the forward primer (SEQ ID NO: 10) corresponded to the 5′-end un-transcribed sequence upstream of the mouse c-mpl gene. The reverse primer (SEQ ID NO: 11) corresponded to an exon 2 sequence where human and mouse genes are identical. Since the human cDNA KI mouse lacks introns, the PCR product of KI mice is shorter than that of the wild-type mouse mpl genomic transcript. Thus the homozygote human cDNA KI mouse yields a PCR product of 309 bp while the wild-type mouse yields a PCR product of 462 bp. The heterozygote mouse yields both PCR products since it carries both human cDNA and mouse genomic DNA alleles.

The template genomic DNA was purified from mouse tails and PCR was carried out using standard protocols. As shown in FIG. 2B, the length of the PCR products was consistent with the prediction, indicating that human c-mpl cDNA was successfully knocked into the mouse genome.

While eltrombopag holds great promise in post-radiation bone marrow recovery, development of eltrombopag for countermeasure of acute radiation syndrome represents a great challenge. This is due to the strict species-specificity of eltrombopag, namely that the molecule binds to the TPO receptors of only humans and chimpanzees. The transmembrane portion of the human and chimpanzee TPO receptors has an amino acid at position 499 (His 499 residue) that is different from all other non-human primates or mammals except chimpanzees, thus leading to the strict species-specificity. This means that traditional animal experimental models will not work for testing eltrombopag, due to ineffective drug binding to the TPO receptors of alternative non-human primates or mammals, thus a resultant lack of response to eltrombopag. However, the TPO receptor (c-Mpl) cDNA knock-in mouse Mpl^{hmMPL} described in Examples 1 and 2 above provides an ideal animal model to test the effectiveness of eltrombopag and other TPO mimetics as therapeutic interventions for acute radiation syndrome.

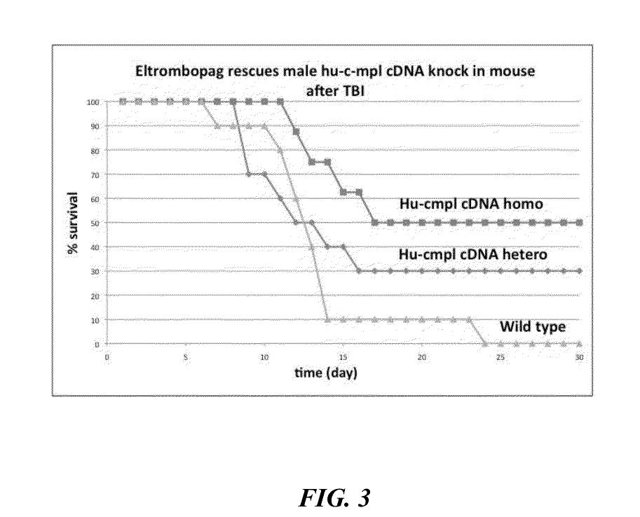

FIG. 3 shows the effects of eltrombopag on the rescue of mouse survival after lethal total body irradiation of Mpl^{hmMPL} knock-in mice. All mice received 8 Gy total body irradiation and were treated with eltrombopag 24 hours after irradiation. The homozygous of Mpl^{hmMPL} knock-in mice survived longer than the heterozygous transgenic mice, while the heterozygous mice survived longer than the wild type. It is believed that eltrombopag facilitates bone marrow recovery after radiation injury to the hematopoietic system.

### Example 3

### Sequence Design and Construction of Human TPO Receptor (c-mpl) Exon 10 Knock-in MouseMpl^{hExon10}

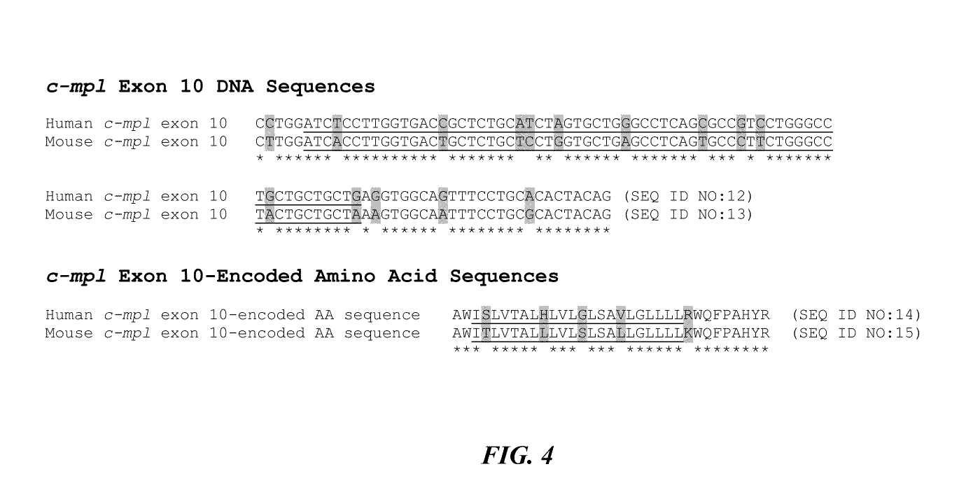

For both human and mouse, the trans-membrane domain (TM) of the TPO receptor (c-Mpl) is encoded by exon 10 of the c-mpl gene. The DNA sequences of human and mouse exon 10 are aligned in FIG. 4. The alignment reveals that the two genes are highly homologous; however, there are a total of 15 base pairs that are different. Human exon 10 sequence was used as a cassette for inserting into the mouse genome to replace its mouse counterpart sequence as described below, resulting in an c-mpl exon 10 mouse knock-out/human knock-in mouseMpl^{hExon10}. The rest of the mouse c-mpl gene remains intact.

Alignment of the exon 10-encoded amino acid sequences of the two species revealed that 5 amino acids are different between them, four of them being in the trans-membrane domain. Thus the c-mpl exon 10 mouse knock-out/human knock-in mouse generated produces a TPO receptor (c-Mpl) with exactly the same amino acid sequence except these five amino acids of human version.

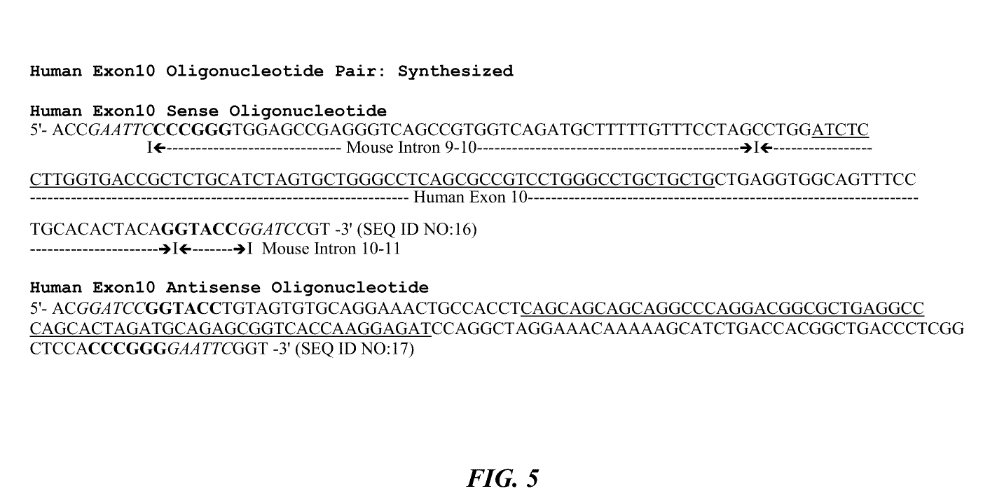

Human exon10 sense and antisense oligonucleotides with flanking sequences corresponding to mouse introns 9-10 and 10-11, respectively, were synthesized, annealed and subcloned as a 169 bp fragment into EcoRI and BamHI sites of plasmid pBluescript SK vector (FIG. 5). The SmaI and KpnI fragment containing the synthetic human Exon10 and the flanking mouse intron sequences were used to replace the mouse SmaI-Exon10-KpnI sequences.

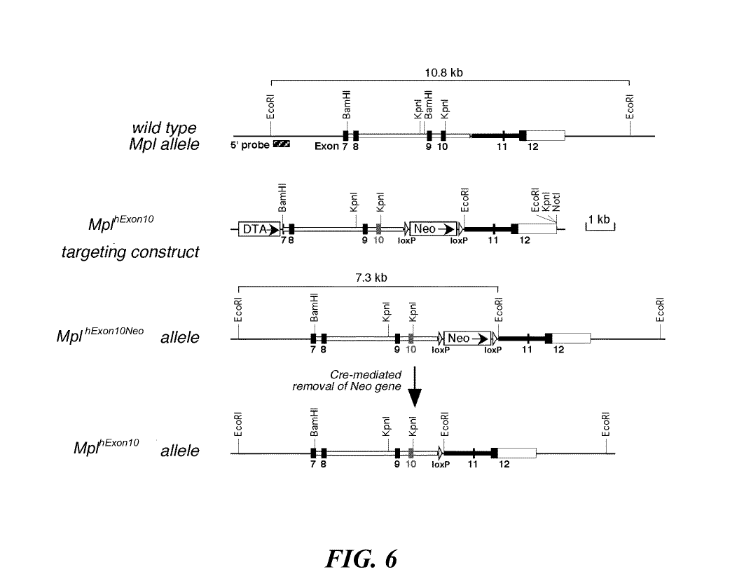

To create Mpl knock-in mice, the entire mouse c-mpl exon 10 (encoding the amino acids 489-521 of SEQ ID NO: 3) was replaced with human c-mpl exon 10 (encoding the amino acids 490-522 of SEQ ID NO: 1). The Mpl^{hExon10}knock-in construct were generated by inserting 3.4 kb c-mpl 5′ flanking genomic DNA containing mouse c-mpl exons 7-9 and human mpl exon 10, and 3.0 kb 3′ sequence containing mouse c-mpl exon 11-12 into the 5′ and 3′ multiple cloning sites of pKII lox vector at the BamHI-XhoI and EcoRI sites, respectively (FIG. 6). Similarly, NotI-linearized Mpl^{hExon10} targeting construct was electroporated into W4 mouse ES cells and positive targeted ES cells were obtained and confirmed by Southern blotting genotyping. Mpl^{hExon10Neo} chimera and heterozygous mice were generated from the targeted ES cells. By breeding Mpl^{hExon10Neo} chimeras with Tg(CMV-cre)1Cgn/J, the neomycin resistant gene cassette was removed to create Mpl^{hExon10} mice.

### Example 4

### Experimental Verification of Human TPO Receptor (c Mpl)Exon 10 Knock-in MouseMpl^{hExon10} at the DNA Level

To experimentally verify that human exon 10 sequence was successfully knocked into the mouse genome to replace the mouse exon 10, genomic DNA PCR was carried out using human- and mouse-specific primers. The forward primers corresponded to human (5′-GCTCTGCATCTAGTGCT-3′ SEQ ID NO: 26) and mouse (5′-CTACTGCTGCTAAAGTGG-3′ SEQ ID NO: 27) exon 10 sequences. For clarity, two reverse mouse primers were used, each pairing with a species-specific forward primer but both located in the antisense strand of mouse intron 10-11 immediately downstream of exon 10 (wild-type mouse reverse primer 3′-CAGTAAGGCTGAGTCCTTTC-5′ (SEQ ID NO: 28) and KI mouse reverse Primer 3′-GGACAGACCTTATAGGAG-5′ (SEQ ID NO: 29)). Thus the homozygote human exon 10 knock-in mice yields a PCR product of 656 bp only with human forward and KI mouse reverse primers, while wild-type mice yield a PCR product of 365 bp only with mouse forward and wild-type mouse reverse primers. Heterozygote mice would yield both PCR products since it would carried both human and mouse alleles of exon 10.

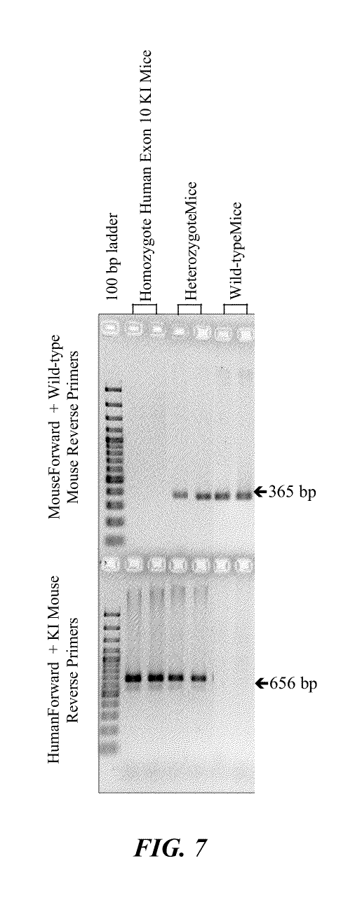

The template genomic DNA was purified from mouse tails and standard PCR protocols were employed to generate PCR products. As shown in FIG. 7, the species specificities of the PCR reactions and the lengths of the PCR products were consistent with the prediction, indicating that exon 10 of human c-mpl was successfully knocked into the mouse genome to replace its mouse counterpart.

### Example 5

### Experimental Verification of Human TPO Receptor (c-mpl) Exon 10 Knock-in Mpl^{hExon10}Mouse at the RNA Level

To confirm that the c-mpl human exon10 KI mouse expresses the human version of exon 10, reverse-transcriptase (RT)-PCR was carried out using mouse- and human-specific primers. The human forward primer is located in the sequence corresponding to the trans-membrane domain (5′ TGACCGCTCTGCATCTA; SEQ ID NO:30). The mouse forward primer differs from the human forward primer in four bases (5′TGACTGCTCTGCTCCTG; SEQ ID NO:31). Under the right conditions the mouse primer only amplifies mouse RNA (or cDNA) and human primer only human RNA (or cDNA). The common reverse primer is located in the sequence of mouse exon 11 and is an anti-sense strand sequence (3′-CATGGAGTCTCTGTGACG-5′; SEQ ID NO:32). The PCR product is 157 bp in length.

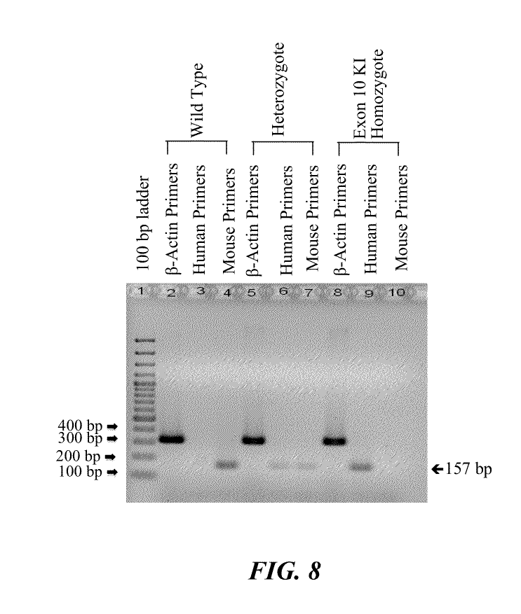

Total RNA was extracted from mouse bone marrow and RT-PCR performed using standard protocols. As shown in FIG. 8, the mouse primer set amplified only the RNA preparations from wild-type (WT) and heterozygote mice and the human primer set amplified only the RNA preparations from homozygote knock-in and heterozygote mice. The PCR product was of the correct size (˜160 bp). These results confirm that the knock-in mice carry the human version of exon 10 of c-mpl, which is transcribed correctly.

### Example 6

### Experimental Verification of Human TPO Receptor (c-mpl) Exon 10 Knock-in MouseMpl^{hExon10} at the RNA Level (cDNA Sequencing)

The PCR results shown in Examples 4 and 5 indicate that human exon 10 of c-mpl was successfully knocked into the mouse genome. To verify that the KI mouse indeed carries the exact human version of exon 10 sequence and that it is correctly transcribed and spliced, cDNA sequencing was carried out.

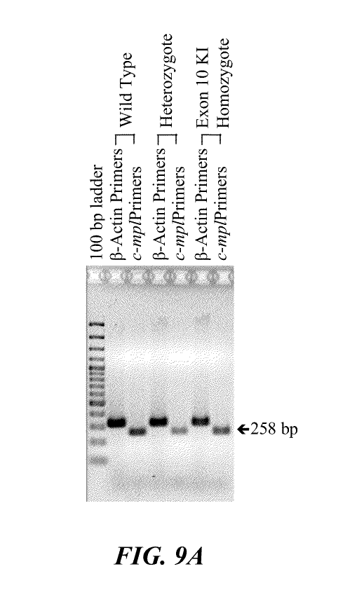

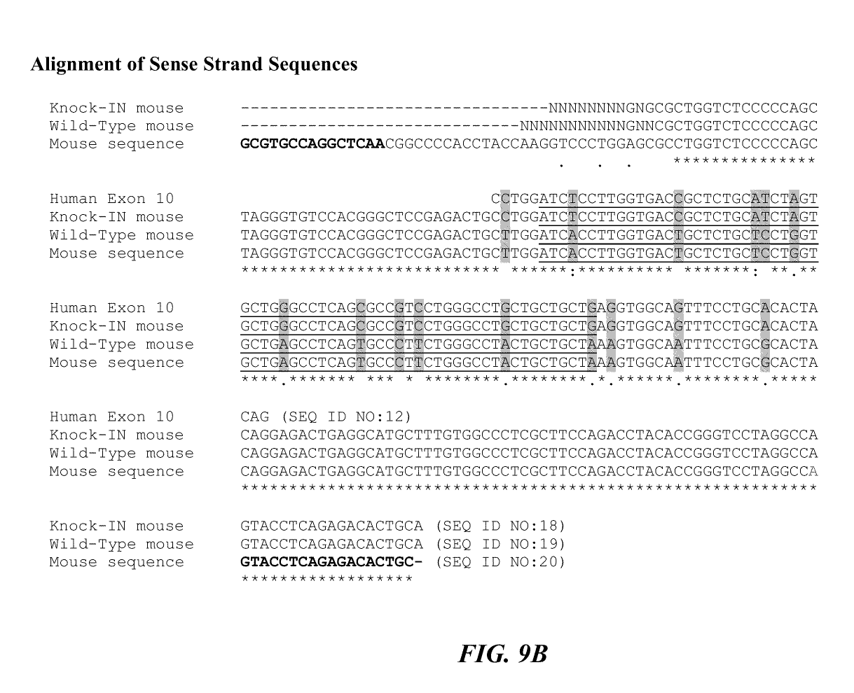

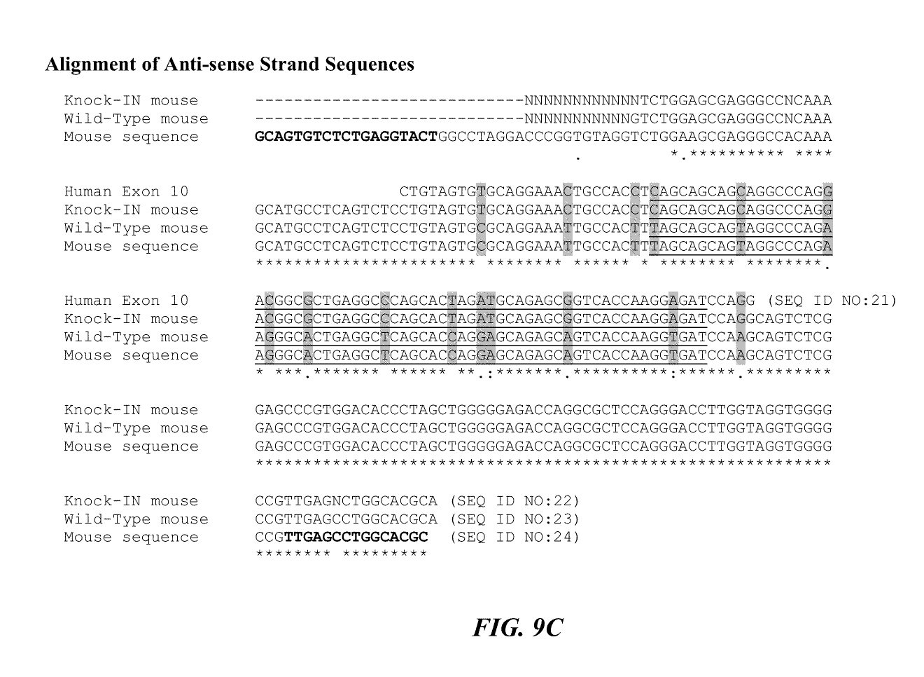

Total RNA was extracted from mouse bone marrow and cDNA synthesized using random primers following standard protocols. cDNA was amplified using the primers corresponding to the mouse sequences flanking exon 10. The forward primer (5′-GCGTGCCAGGCTCAA-3′; SEQ ID NO:33) is located in exon 9 and the reverse primer (5′-TTGAGCCTGGCACGC-3′; SEQ ID NO:34) in exon 11. The cDNA PCR product comprises the entire exon 10 and its flanking regions and is 258 bp in length. As shown in FIG. 9A, the PCR product obtained was around 260 bp as expected. The PCR products of both wild-type and knock-in mice were subject to DNA sequencing in both strands using the PCR primers also as sequencing primers. The sense-strand sequences are aligned in FIG. 9B, which shows that: 1) the wild-type mouse carries the mouse sequence in the entire region as expected; and 2) the knock-in mouse carries the mouse sequence except that of exon 10, where it is replaced by the human sequence. The sequencing results also show that all 15 nucleotide mismatches between the human and mouse exon 10 sequences (as highlighted in FIG. 4) have been changed to the human version in the KI mouse. The sequencing results of the anti-sense strands (FIG. 9C) are consistent with those of the forward strands, proving that human exon 10 sequence has been correctly knocked into the KI mouse's c-mpl gene to replace its mouse counterpart.

### Example 7

### Eltrombopag Treatment of Mpl^{hExon10} KI Mice Increases Platelet and Bone Marrow Cell Populations

Mpl^{hExon10} KI mice (9 to 13 weeks old, male and female) were fed with 25 mg eltrombopag (ePag)/kg/day or vehicle by gavage for 15 days. Eltrombopag is a non-peptide mimetic of the TPO receptor. Mice were sacrificed on the 16^{th} day for cell analysis.

To examine platelet levels, whole blood was obtained by heart puncture and CBC was done by Heska HemaTrue Hematology analyzer. Whole blood was also stained by anti CD41 and anti CD61 antibodies. The ratio of platelets to RBCs was measured by flow cytometry after whole blood was stained with anti-CD41 and anti-CD61 antibodies. The count of platelets was calculated by multiplying the ratio with the count of RBCs obtained by Heska HemaTrue Hematology Analyzer. The data is presented as mean±standard error of the mean.

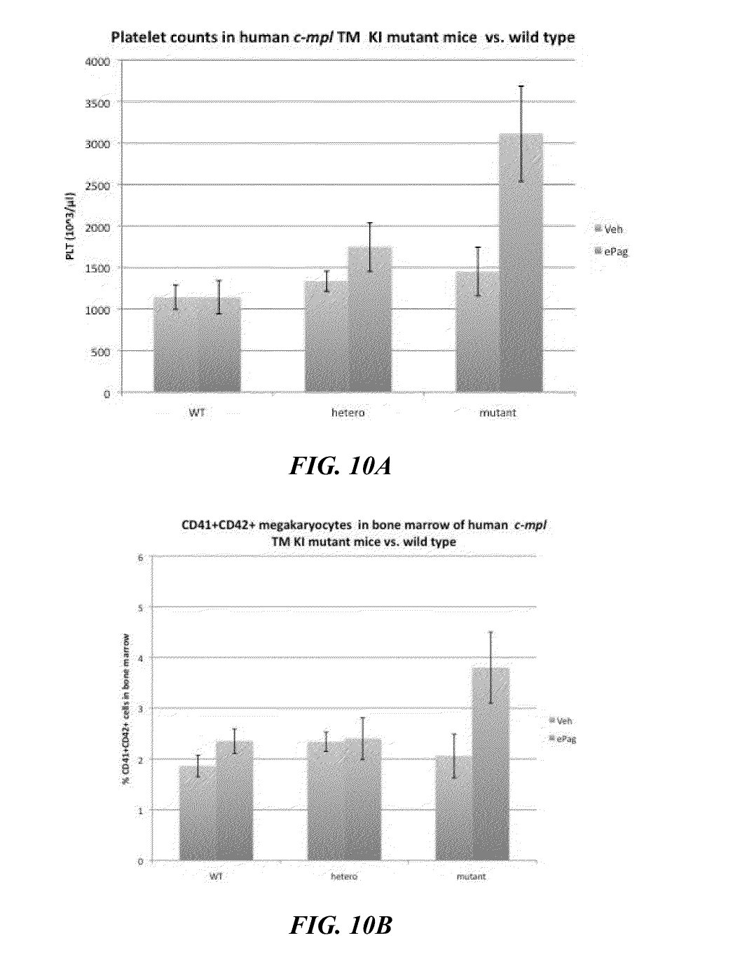

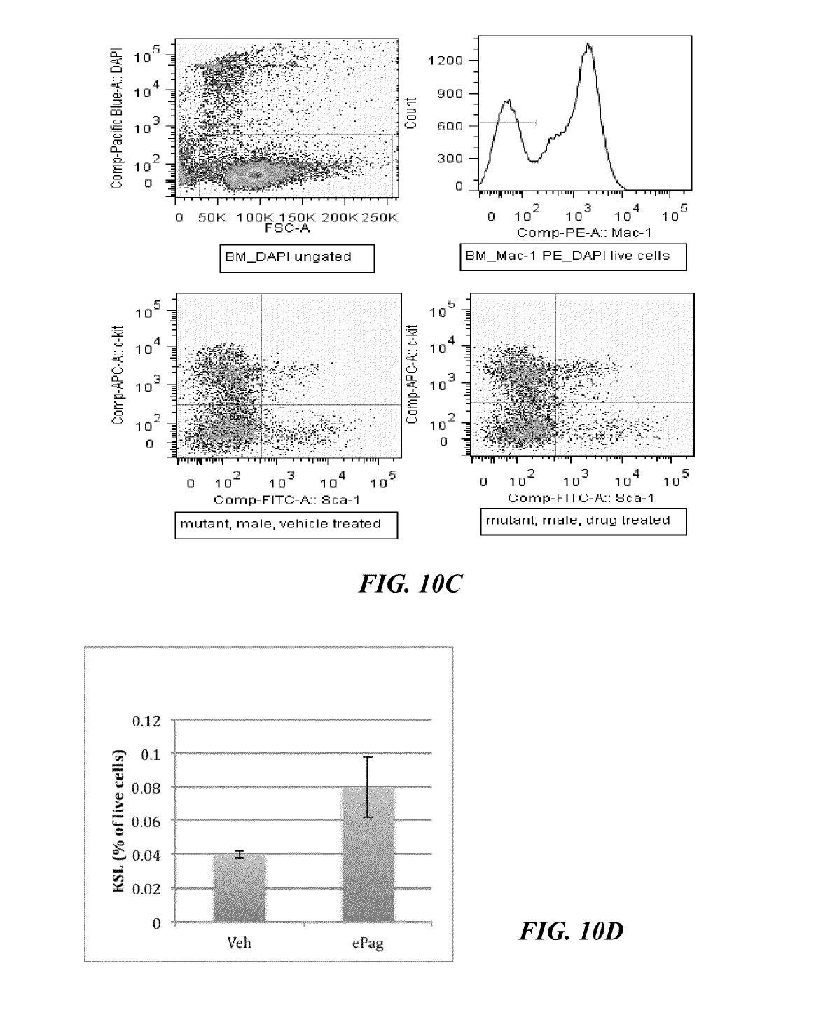

As shown in FIG. 10A, eltrombopag significantly increases the platelet counts in the peripheral blood of the homozygous human TPO receptor (c-mpl) exon 10 knock-in mice Mpl^{hExon10}(mutant). It is noteworthy that the baseline (i.e., no eltrombopag treatment) platelet count in KI mouse is not significantly different from that of the wild-type.

To examine bone marrow cell populations, mice were sacrificed on day 16 and bone marrow cells were flushed out of the femur and tibia. RBCs were lysed by ACK buffer. The bone marrow mononuclear cells were stained with anti-CD41-PE and anti-CD42-APC. DAPI was added before flow cytometric analysis to gate away dead cells. The number of mice in each genotype is 5-7. All results are shown as the mean±standard error of the mean. As shown in FIG. 10B, eltrombopag significantly increased the bone marrow CD41⁺CD42⁺ cells in the homozygous Mpl^{hExon10}KI mice (mutant).

In a separate experiment, bone marrow mononuclear cells were stained with anti-lineages-PE (Gr-1, Mac-1, B220, Ter119, CD4 and CD8), anti-Sca-1 FITC, and anti-c-kit-APC. DAPI-pacific blue was added before flow cytometric analysis to eliminate dead cells. To analyze KSL (linage⁻, c-ki⁺, sca⁺) population, live cells (DAPI-) are first gated, followed by gating the linage⁻ population and selecting c-kit⁺/sca-1⁺ population within the linage⁻ population. FIGS. 10C and 10D show that eltrombopag significantly increased the bone marrow Lin⁻KSL (stem) cells in the Mpl^{hExon10}KI (mutant).

### Example 8

### Eltrombopag Improves Survival of Irradiated Human c-mpl TM (Exon 10) Knock-in Mice

A pilot radiation study of was performed using human c-mpl TM KI mice before completion of backcrossing to >95% C57B1. Male human c-mpl TM (exon 10) KI mice received 7.75 Gy TBI. Twenty-four hours after irradiation, mice were gavaged with either vehicle (0 mg/kg of eltrombopag) or one of the three doses of eltrombopag (12.5 mg/kg, 25 m g/kg, vs. 50 mg/kg daily for 15 days (n=8-10) for each experimental group.

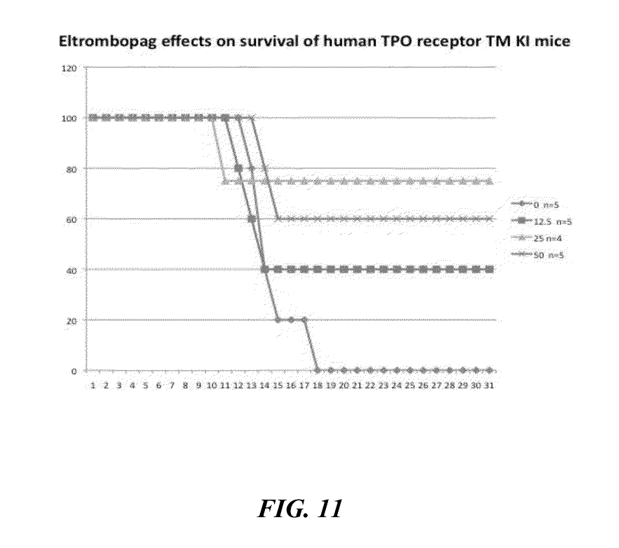

All mice treated with vehicle died around day 19 as shown in the survival curve of FIG. 11. Eltrombopag improved the survival of mice and it appeared that higher doses (25 mg/kg and 50 mg/kg) yielded better survival. Note that the study was conducted with mice at earlier stage of development. Nevertheless, this data indicates that eltrombopag improves the survival of human c-mpl KI mice after TBI of homozygous mutants.

### Example 9

### Eltrombopag Promotes Differentiation of Megakaryocytes and CD41+ CD34− Cells in Irradiated Ex Vivo Human Bone Marrow Culture in 3D Bioreactor

Human bone marrow mononuclear cells were inoculated into the 6-well bioreactor as described above (3.5×10⁶ cells in 0.6 mL). Megakaryocyte differentiation was induced by an addition of TPO (5 ng/mL) and IL11 (5 ng/mL) to the serum free IMDM medium supplemented with 2 mM L-Glutamine, 25 mM HEPES, 10⁻⁴ M β-mercaptoethanol, 10⁻⁶M hydrocortisone, 0.8% of Penicillin/Streptomycin solution (10,000 U/mL penicillin, 10,000 μg/mL Streptomycin), 20% BIT 9500 serum substitute and 3% of human serum. Three independent cultures were set up for each experiment and maintained for 3 weeks. Cultures were irradiated on day 6 or 12 using a Cs-137 source at the dose rate 3.2 Gy/min. After removal of TPO from the media two doses of eltrombopag, 8 and 12 μg/mL have been added to the cultures 24 h after irradiation and daily thereafter. Cultures were screened weekly for cell viability (Trypan blue exclusion test) and for megakaryocytes production expressed as number of megakaryocytes per 1000 bone marrow cells on Wright stained cytospin slides.

Flow cytometry analysis of CD41+CD34− cells (marker for precursor/progenitor of thrombopoiesis) was also performed for megakaryocytes and progenitors as an additional experimental end-point. 10⁵ cells were washed with washing buffer (2% FBS in DPBS), blocked with mouse serum for 20 min and stained with mouse anti human CD41PE and mouse anti human CD 34 FITC antibodies (BD Pharmingen, San Diego, Calif.) for 30 min. Cells were analyzed on FACS- Calibur Flow Cytometer (Becton-Dickinson, Rockville, Md.). CD41+CD34− cell population was gated as a subpopulation of all living cells analyzed.

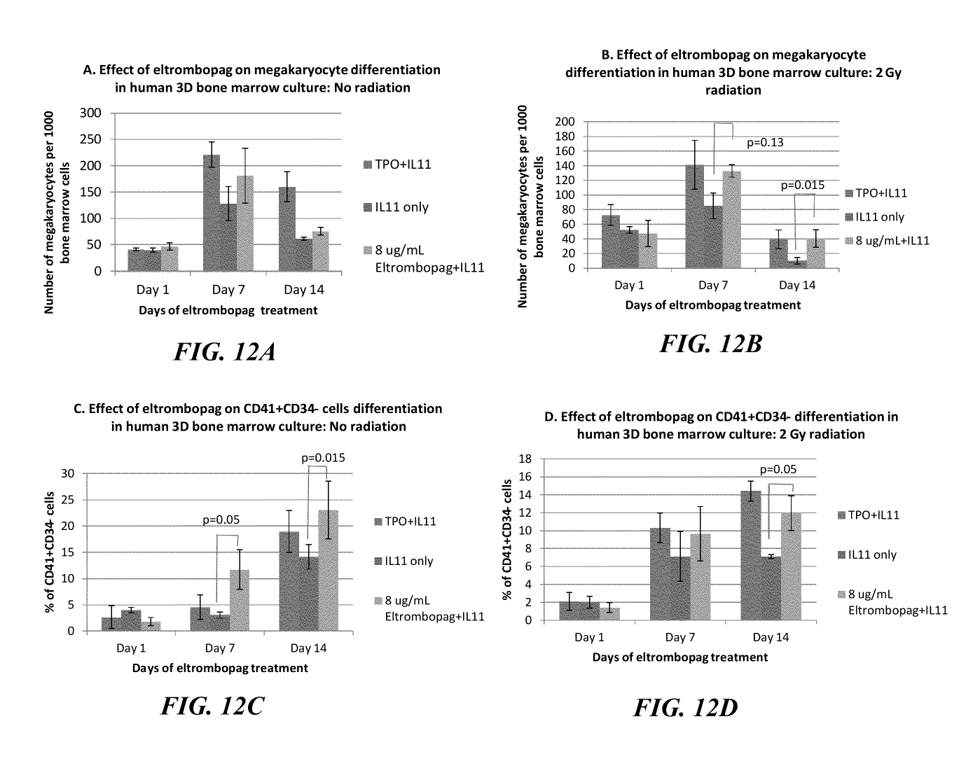

Human 3D bone marrow mononuclear cells were treated with TPO and IL11 (5 ng/mL each) for 6-7 days to induce megakaryocyte differentiation. Culture media was replaced with cultures containing IL-11 only (red bars), vs. TPO+IL11 (blue bars) vs. eltrombopag (8 μg/mL)+IL11 for the groups of control cultures (FIGS. 12A, 12C) and the group of irradiated cultures (FIGS. 12B, 12D). The cultures were maintained for another 14 days and screened for the presence of megakaryocytes and CD41+CD34− cells (precursors/progenitors for thrombopoiesis) every 7 days. Each culture was set up in three replicates. The data show that eltrombopag performed as well as TPO in promoting megakaryocytes and in promoting CD41+CD34− cells in both the non-irradiated control cultures and in the irradiated cultures.

### Example 10

### Eltrombopag Improves Blood Cell Recovery in Irradiated Human c-mpl TM (Exon 10) Knock-in Mice

TM KI homozygote mutant mice (11 weeks old, male and female) were treated with 6.5 Gy TBI and at 24 hr post IR fed with 25 mg ePag/kg/day or vehicle by oral gavage daily for 28 days or till sacrificed. Mice were sacrificed on the day specified. Whole blood was obtained by heart puncture and CBC was done by Heska HemaTrue Hematology analyzer. Whole blood was also stained by anti CD41 and anti CD61 antibodies. BM was extracted and stained with mCD41 and mCD42d for flow cytometry analysis of CD41− CD42d+ cells (markers for precursor and progenitors for thrombopoiesis).

The ratio of platelets to RBCs was measured by flow cytometry after whole blood was stained with anti-CD41 and anti-CD61 antibodies. The count of platelets was calculated by multiplying the ratio with the count of RBCs obtained by Heska HemaTrue Hematology Analyzer. Week 0 is normal mice without IR and gavage.

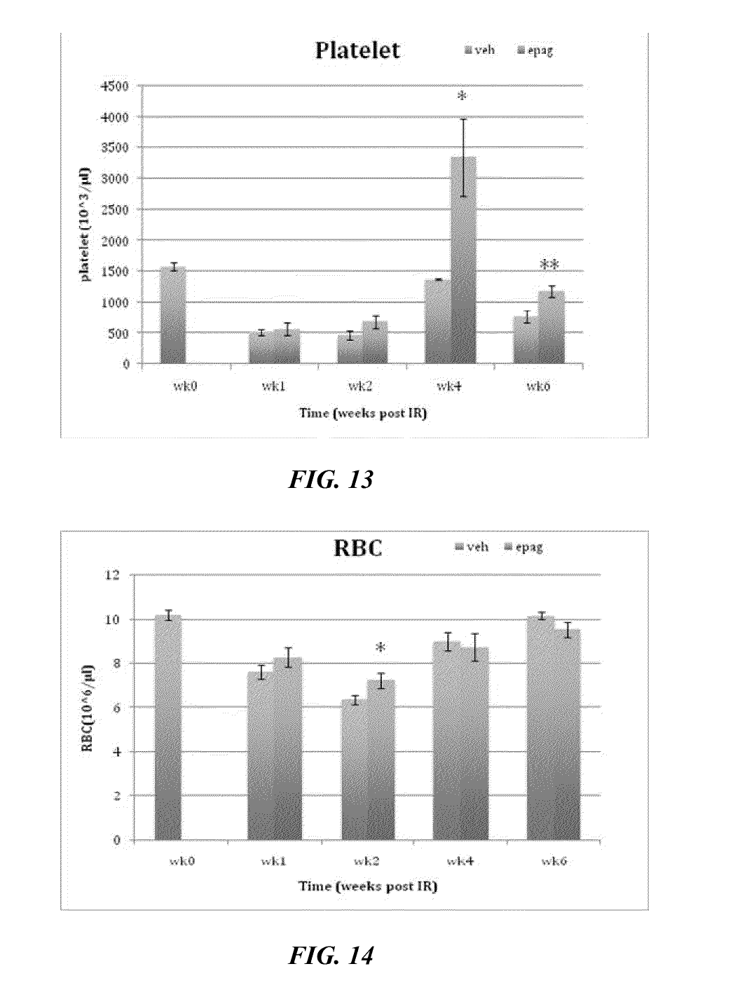

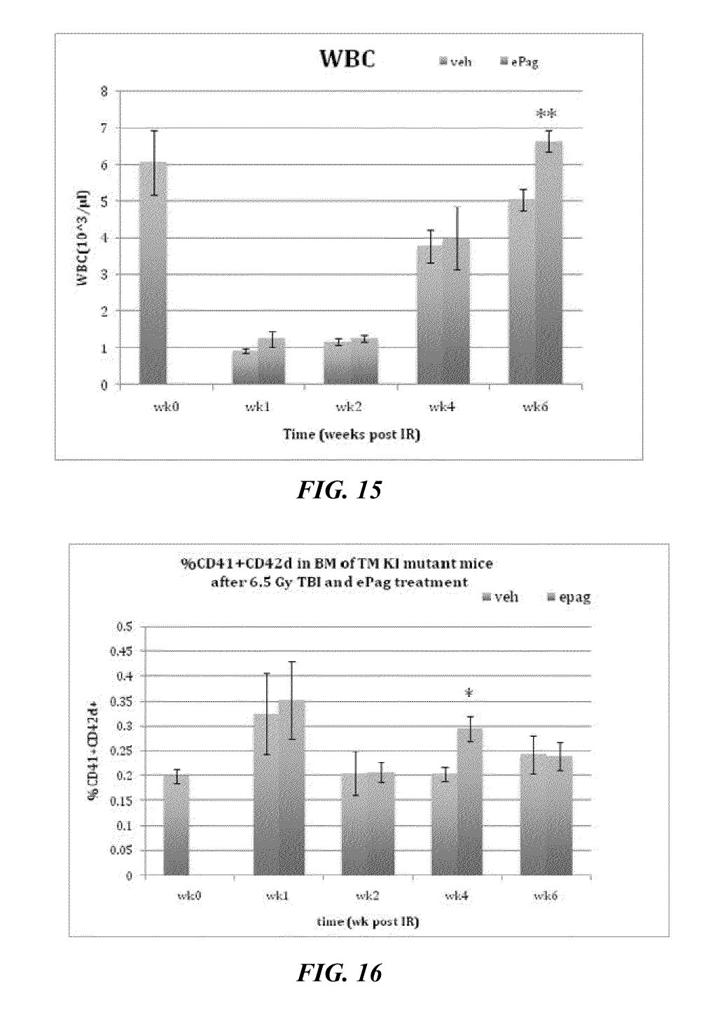

The results of the analysis are presented in FIGS. 13-16, which illustrate a significant improvement in platelet counts 4 and 6 weeks post IR (FIG. 13), an improvement in red blood cell counts 2 weeks post IR (FIG. 14), a significant improvement in white blood cell counts at 6 weeks post IR (FIG. 15), and a significant improvement in the count of bone marrow precursor/progenitor cells for thrombopoiesis 4 weeks post IR (FIG. 16). These results offer an explanation for the improved survival rate in treating ARS with Eltrombopag (see FIG. 11).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of treating a subject for acute radiation syndrome comprising:
administering a c-Mpl receptor agonist to the subject under conditions effective to treat acute radiation syndrome.

2. The method of claim 1 further comprising:
selecting a subject that has been exposed to a non-therapeutically high dose of radiation prior to said administering.

3. The method of claim 1 further comprising:
selecting a subject at risk of being exposed to a non-therapeutically high dose of radiation and
carrying out said administering prior to the exposure.

4. The method of claim 1, wherein said subject has radiation hematopoietic syndrome of acute radiation syndrome.

5. The method of claim 1, wherein the c-Mpl receptor agonist comprises a recombinant thrombopoietin protein or peptide thereof.

6. The method of claim 1, wherein the c-Mpl receptor agonist comprises a non-peptide thrombopoietin mimetic.

7. The method of claim 1, wherein the c-Mpl agonist comprises a thrombopoietin peptide mimetic or peptibody.

8. The method of claim 1, wherein the c-Mpl agonist comprises an agonist antibody.

9. The method of claim 1 further comprising:
administering cell therapy, one or more cytokines, or one or more immune modulators prior to, concurrently with, or after said administering the c-Mpl receptor agonist.

10. A method of treating a subject for chronic radiation syndrome comprising:
administering a c-Mpl receptor agonist to the subject under conditions effective to treat chronic radiation syndrome.

11. The method of claim 10 further comprising:
selecting a subject that has been repeatedly exposed to a non-therapeutic dose of radiation prior to said administering.

12. The method of claim 10, wherein the c-Mpl receptor agonist comprises a recombinant thrombopoietin protein or peptide thereof.

13. The method of claim 10, wherein the c-Mpl receptor agonist comprises a non-peptide thrombopoietin mimetic.

14. The method of claim 10, wherein the c-Mpl agonist comprises a thrombopoietin peptide mimetic or peptibody.

15. The method of claim 10, wherein the c-Mpl agonist comprises an agonist antibody.

16. The method of claim 10 further comprising:
administering cell therapy, one or more cytokines, or one or more immune modulators prior to, concurrently with, or after said administering the c-Mpl receptor agonist.

17. A method of treating a subject having a bone marrow injury resulting from exposure to a non-therapeutic chemical agent comprising:
administering a c-Mpl receptor agonist to the subject under conditions effective to treat the bone marrow injury resulting from exposure to the non-therapeutic chemical agent.

18. The method of claim 17, wherein the non-therapeutic chemical agent is selected from 2,2,-dichlordiethyl sulfide (mustard gas), pinacolyl methylphosphono-fluoridate (nerve gas), and nitrogen mustard.

19. The method of claim 17 further comprising:
selecting a subject that has been exposed to the non-therapeutic chemical agent prior to said administering.

20. The method of claim 17 further comprising:
selecting a subject at risk of being exposed to the non-therapeutic chemical agent and
carrying out said administering prior to the exposure.


SEQUENCE LISTING (34 sequences)

SEQ ID NO: 1 - protein, 635 aa, Homo sapiens
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 2 - protein, 23 aa, Artificial
  Humanized Thrombopoietin Receptor Transmembrane Domain
IXLVTALLHLVLXLSAXLGLLLL

SEQ ID NO: 3 - protein, 633 aa, Mouse
MPSWALFMVTSCLLLALPNQAQVTSQDVFLLALGTEPLNCFSQTFEDLTCFWDEEEAAPSGTYQLLYAYRGEKPRACPLYSQSVPTFGTRYVCQFPAQDEVRLFFPLHLWVKNVSLNQTLIQRVLFVDSVGLPAPPRVIKARGGSQPGELQIHWEAPAPEISDFLRHELRYGPTDSSNATAPSVIQLLSTETCCPTLWMPNPVPVLDQPPCVHPTASQPHGPVRTSPAGEAPFLTVKGGSCLVSGLQAGKSYWLQLRSQPDGVSLRGSWGPWSFPVTVDLPGDAVTIGLQCFTLDLKMVTCQWQQQDRTSSQGFFRHSRTRCCPTDRDPTWEKCEEEEPRPGSQPALVSRCHFKSRNDSVIHILVEVTTAQGAVHSYLGSPFWIHQAVLLPTPSLHWREVSSGRLELEWQHQSSWAAQETCYQLRYTGEGREDWKVLEPSLGARGGTLELRPRARYSLQLRARLNGPTYQGPWSAWSPPARVSTGSETAWITLVTALLLVLSLSALLGLLLLKWQFPAHYRRLRHALWPSLPDLHRVLGQYLRDTAALSPSKATVTDSCEEVEPSLLEILPKSSESTPLPLCPSQPQMDYRGLQPCLRTMPLSVCPPMAETGSCCTTHIANHSYLPLSYWQQP

SEQ ID NO: 4 - protein, 633 aa, Artificial
  Humanized mouse thrombopoietin receptor
MPSWALFMVTSCLLLALPNQAQVTSQDVFLLALGTEPLNCFSQTFEDLTCFWDEEEAAPSGTYQLLYAYRGEKPRACPLYSQSVPTFGTRYVCQFPAQDEVRLFFPLHLWVKNVSLNQTLIQRVLFVDSVGLPAPPRVIKARGGSQPGELQIHWEAPAPEISDFLRHELRYGPTDSSNATAPSVIQLLSTETCCPTLWMPNPVPVLDQPPCVHPTASQPHGPVRTSPAGEAPFLTVKGGSCLVSGLQAGKSYWLQLRSQPDGVSLRGSWGPWSFPVTVDLPGDAVTIGLQCFTLDLKMVTCQWQQQDRTSSQGFFRHSRTRCCPTDRDPTWEKCEEEEPRPGSQPALVSRCHFKSRNDSVIHILVEVTTAQGAVHSYLGSPFWIHQAVLLPTPSLHWREVSSGRLELEWQHQSSWAAQETCYQLRYTGEGREDWKVLEPSLGARGGTLELRPRARYSLQLRARLNGPTYQGPWSAWSPPARVSTGSETAWISLVTALHLVLGLSAVLGLLLLRWQFPAHYRRLRHALWPSLPDLHRVLGQYLRDTAALSPSKATVTDSCEEVEPSLLEILPKSSESTPLPLCPSQPQMDYRGLQPCLRTMPLSVCPPMAETGSCCTTHIANHSYLPLSYWQQP

SEQ ID NO: 5 - DNA, 1902 bp, Artificial
  Humanized mouse TPO receptor
atgccctcttgggccctcttcatggtcacctcctgcctcctcttggcccttccaaaccaggcacaagtcaccagccaagatgtcttcttgctggccttgggcacagagcccctgaactgcttctcccaaacatttgaggacctcacctgcttctgggatgaggaagaggcagcacccagtgggacataccagctgctgtatgcctaccgaggagagaagccccgtgcatgccccctgtattcccagagtgtgcccacctttggaacccggtatgtgtgccagtttccagcccaggatgaagtgcgcctcttctttccgctgcacctctgggtgaagaatgtgtccctcaaccagactttgatccagcgggtgctgtttgtggatagtgtgggcctgccagctccccccagggtcatcaaggccaggggtgggagccaaccaggggaacttcagatccactgggaggcccctgctcctgaaatcagtgacttcctgaggcatgaactccgctatggccccacggactccagcaacgccactgccccctccgtcattcagctgctctccacagaaacctgctgccccactttgtggatgccgaacccagtccctgttcttgaccagcctccgtgtgttcatccgacagcatcccaaccgcatggaccagtgaggacctccccagctggagaagctccatttctgacagtgaagggtggaagctgtctcgtctcaggcctccaggctggcaaatcctactggctccagctacgcagccaacccgacggggtctcccttcgtggctcctggggaccctggtccttccctgtgactgtggatcttccaggagatgcagtgacaattggacttcagtgctttaccttggatctgaagatggtcacctgccagtggcagcaacaagaccgcactagctcccaaggcttcttccgccacagcaggacgaggtgctgccccacagacagggaccccacctgggagaaatgtgaagaggaggaaccgcgtccaggatcacagcccgctctcgtctcccgctgccacttcaagtcacgaaatgacagtgttattcacatccttgtagaggtgaccacagcgcaaggtgccgttcacagctacctgggctcccctttttggatccaccaggctgtgctccttcccaccccgagcctgcactggagggaggtctcaagtggaaggctggagttggagtggcagcaccagtcatcttgggcagctcaagagacctgctaccagctccggtacacgggagaaggccgtgaggactggaaggtgctggagccatctctcggtgcccggggagggaccctagagctgcgcccccgagctcgctacagcttgcagctgcgtgccaggctcaacggccccacctaccaaggtccctggagcgcctggtctcccccagctagggtgtccacgggctccgagactgcctggatctccttggtgaccgctctgcatctagtgctgggcctcagcgccgtcctgggcctgctgctgctgaggtggcagtttcctgcacactacaggagactgaggcatgctttgtggccctcgcttccagacctacaccgggtcctaggccagtacctcagagacactgcagccctaagtccttctaaggccacggttaccgatagctgtgaagaagtggaacccagcctcctggaaatcctccctaagtcctcagagagcactcctttacctctgtgtccctcccaacctcagatggactacagaggactgcaaccttgcctgcggaccatgcccctgtctgtgtgtccacccatggctgagacggggtcctgctgcaccacacacattgccaaccactcctacctaccactaagctattggcagcagccctga

SEQ ID NO: 6 - protein, 634 aa, Artificial
  Humanized mouse thrombopoietin receptor
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLKWQFPAHYRRLRHALWPSLPDLHRVLGQYLRDTAALSPSKATVTDSCEEVEPSLLEILPKSSESTPLPLCPSQPQMDYRGLQPCLRTMPLSVCPPMAETGSCCTTHIANHSYLPLSYWQQP

SEQ ID NO: 7 - DNA, 1905 bp, Artificial
  Humanized mouse thrombopoietin receptor
atgccctcctgggccctcttcatggtcacctcctgcctcctcctggcccctcaaaacctggcccaagtcagcagccaagatgtctccttgctggcatcagactcagagcccctgaagtgtttctcccgaacatttgaggacctcacttgcttctgggatgaggaagaggcagcgcccagtgggacataccagctgctgtatgcctacccgcgggagaagccccgtgcttgccccctgagttcccagagcatgccccactttggaacccgatacgtgtgccagtttccagaccaggaggaagtgcgtctcttctttccgctgcacctctgggtgaagaatgtgttcctaaaccagactcggactcagcgagtcctctttgtggacagtgtaggcctgccggctccccccagtatcatcaaggccatgggtgggagccagccaggggaacttcagatcagctgggaggagccagctccagaaatcagtgatttcctgaggtacgaactccgctatggccccagagatcccaagaactccactggtcccacggtcatacagctgattgccacagaaacctgctgccctgctctgcagaggcctcactcagcctctgctctggaccagtctccatgtgctcagcccacaatgccctggcaagatggaccaaagcagacctccccaagtagagaagcttcagctctgacagcagagggtggaagctgcctcatctcaggactccagcctggcaactcctactggctgcagctgcgcagcgaacctgatgggatctccctcggtggctcctggggatcctggtccctccctgtgactgtggacctgcctggagatgcagtggcacttggactgcaatgctttaccttggacctgaagaatgttacctgtcaatggcagcaacaggaccatgctagctcccaaggcttcttctaccacagcagggcacggtgctgccccagagacaggtaccccatctgggagaactgcgaagaggaagagaaaacaaatccaggactacagaccccacagttctctcgctgccacttcaagtcacgaaatgacagcattattcacatccttgtggaggtgaccacagccccgggtactgttcacagctacctgggctcccctttctggatccaccaggctgtgcgcctccccaccccaaacttgcactggagggagatctccagtgggcatctggaattggagtggcagcacccatcgtcctgggcagcccaagagacctgttatcaactccgatacacaggagaaggccatcaggactggaaggtgctggagccgcctctcggggcccgaggagggaccctggagctgcgcccgcgatctcgctaccgtttacagctgcgcgccaggctcaacggccccacctaccaaggtccctggagctcgtggtcggacccaactagggtggagaccgccaccgagaccgcctggatctccttggtgaccgctctgcatctagtgctgggcctcagcgccgtcctgggcctgctgctgctgaagtggcaatttcctgcgcactacaggagactgaggcatgctttgtggccctcgcttccagacctacaccgggtcctaggccagtacctcagagacactgcagccctaagtccttctaaggccacggttaccgatagctgtgaagaagtggaacccagcctcctggaaatcctccctaagtcctcagagagcactcctttacctctgtgtccctcccaacctcagatggactacagaggactgcaaccttgcctgcggaccatgcccctgtctgtgtgtccacccatggctgagacggggtcctgctgcaccacacacattgccaaccactcctacctaccactaagctattggcagcagccctga

SEQ ID NO: 8 - DNA, 480 bp, Mouse
ggctgtatctgacaggaacctgaggggctggccccgggggggattggggcccagcttcctgaagggaggatgggctaaggcaggcacacagtgccggagaagatgccctcttgggccctcttcatggtcacctcctgcctcctcttggcccttccaaaccaggcacaagtcaccagccaaggtgaggtggatagagggtggatgttacctattcacaggcaaagggagccctgggaggggatatggggtaagagactctcactggttcccttcttttacccaacaaacatgactggaatgatcgagagcccaactcaccatctctgttctcagatgtcttcttgctggccttgggcacagagcccctgaactgcttctcccaaacatttgaggacctcacctgcttctgggatgaggaagaggcagcacccagtgggacataccagctgctgtatgcctaccgagggtaggttctgggct

SEQ ID NO: 9 - DNA, 254 bp, Homo sapiens
gaagggaggatgggctaaggcaggcacacagtggcggagaagatgccctcctgggccctcttcatggtcacctcctgcctcctcctggcccctcaaaacctggcccaagtcagcagccaagatgtctccttgctggcatcagactcagagcccctgaagtgtttctcccgaacatttgaggacctcacttgcttctgggatgaggaagaggcagcgcccagtgggacataccagctgctgtatgcctacccgcg

SEQ ID NO: 10 - DNA, 21 bp, Artificial
  Primer
gctgtatctgacaggaacctg

SEQ ID NO: 11 - DNA, 19 bp, Artificial
  Primer
gtcgacgacatacggatgg

SEQ ID NO: 12 - DNA, 97 bp, Homo sapiens
cctggatctccttggtgaccgctctgcatctagtgctgggcctcagcgccgtcctgggcctgctgctgctgaggtggcagtttcctgcacactacag

SEQ ID NO: 13 - DNA, 97 bp, Mouse
cttggatcaccttggtgactgctctgctcctggtgctgagcctcagtgcccttctgggcctactgctgctaaagtggcaatttcctgcgcactacag

SEQ ID NO: 14 - protein, 33 aa, Homo sapiens
AWISLVTALHLVLGLSAVLGLLLLRWQFPAHYR

SEQ ID NO: 15 - protein, 33 aa, Mouse
AWITLVTALLLVLSLSALLGLLLLKWQFPAHYR

SEQ ID NO: 16 - DNA, 169 bp, Artificial
  Human c-mpl exon 10 sense oligonucleotide
accgaattccccgggtggagccgagggtcagccgtggtcagatgctttttgtttcctagcctggatctccttggtgaccgctctgcatctagtgctgggcctcagcgccgtcctgggcctgctgctgctgaggtggcagtttcctgcacactacaggtaccggatccgt

SEQ ID NO: 17 - DNA, 169 bp, Artificial
  Human c-mpl exon 10 antisense oligonucleotide
acggatccggtacctgtagtgtgcaggaaactgccacctcagcagcagcaggcccaggacggcgctgaggcccagcactagatgcagagcggtcaccaaggagatccaggctaggaaacaaaaagcatctgaccacggctgaccctcggctccacccggggaattcggt

SEQ ID NO: 18 - DNA, 227 bp, Artificial
  c-mpl exon 10 cDNA of knock-in mouse
nnnnnnnngngcgctggtctcccccagctagggtgtccacgggctccgagactgcctggatctccttggtgaccgctctgcatctagtgctgggcctcagcgccgtcctgggcctgctgctgctgaggtggcagtttcctgcacactacaggagactgaggcatgctttgtggccctcgcttccagacctacaccgggtcctaggccagtacctcagagacactgca

SEQ ID NO: 19 - DNA, 230 bp, Artificial
  c-mpl exon 10 cDNA of wild type mouse
nnnnnnnnnnngnncgctggtctcccccagctagggtgtccacgggctccgagactgcttggatcaccttggtgactgctctgctcctggtgctgagcctcagtgcccttctgggcctactgctgctaaagtggcaatttcctgcgcactacaggagactgaggcatgctttgtggccctcgcttccagacctacaccgggtcctaggccagtacctcagagacactgca

SEQ ID NO: 20 - DNA, 258 bp, Mouse
gcgtgccaggctcaacggccccacctaccaaggtccctggagcgcctggtctcccccagctagggtgtccacgggctccgagactgcttggatcaccttggtgactgctctgctcctggtgctgagcctcagtgcccttctgggcctactgctgctaaagtggcaatttcctgcgcactacaggagactgaggcatgctttgtggccctcgcttccagacctacaccgggtcctaggccagtacctcagagacactgc

SEQ ID NO: 21 - DNA, 97 bp, Homo sapiens
ctgtagtgtgcaggaaactgccacctcagcagcagcaggcccaggacggcgctgaggcccagcactagatgcagagcggtcaccaaggagatccagg

SEQ ID NO: 22 - DNA, 231 bp, Artificial
  c-mpl exon 10 cDNA of knock-in mouse
nnnnnnnnnnnntctggagcgagggccncaaagcatgcctcagtctcctgtagtgtgcaggaaactgccacctcagcagcagcaggcccaggacggcgctgaggcccagcactagatgcagagcggtcaccaaggagatccaggcagtctcggagcccgtggacaccctagctgggggagaccaggcgctccagggaccttggtaggtggggccgttgagnctggcacgca

SEQ ID NO: 23 - DNA, 231 bp, Artificial
  c-mpl exon 10 cDNA of wild-type mouse
nnnnnnnnnnngtctggagcgagggccncaaagcatgcctcagtctcctgtagtgcgcaggaaattgccactttagcagcagtaggcccagaagggcactgaggctcagcaccaggagcagagcagtcaccaaggtgatccaagcagtctcggagcccgtggacaccctagctgggggagaccaggcgctccagggaccttggtaggtggggccgttgagcctggcacgca

SEQ ID NO: 24 - DNA, 258 bp, Mouse
gcagtgtctctgaggtactggcctaggacccggtgtaggtctggaagcgagggccacaaagcatgcctcagtctcctgtagtgcgcaggaaattgccactttagcagcagtaggcccagaagggcactgaggctcagcaccaggagcagagcagtcaccaaggtgatccaagcagtctcggagcccgtggacaccctagctgggggagaccaggcgctccagggaccttggtaggtggggccgttgagcctggcacgc

SEQ ID NO: 25 - protein, 18 aa, Artificial
  Thrombopoietin mimetic peptide
XXXXGPTLXXWLXXXXXX

SEQ ID NO: 26 - DNA, 17 bp, Artificial
  Primer
gctctgcatctagtgct

SEQ ID NO: 27 - DNA, 18 bp, Artificial
  Primer
ctactgctgctaaagtgg

SEQ ID NO: 28 - DNA, 20 bp, Artificial
  Primer
cagtaaggctgagtcctttc

SEQ ID NO: 29 - DNA, 18 bp, Artificial
  Primer
ggacagaccttataggag

SEQ ID NO: 30 - DNA, 17 bp, Artificial
  Primer
tgaccgctctgcatcta

SEQ ID NO: 31 - DNA, 17 bp, Artificial
  Primer
tgactgctctgctcctg

SEQ ID NO: 32 - DNA, 18 bp, Artificial
  Primer
catggagtctctgtgacg

SEQ ID NO: 33 - DNA, 15 bp, Artificial
  Primer
gcgtgccaggctcaa

SEQ ID NO: 34 - DNA, 15 bp, Artificial
  Primer
ttgagcctggcacgc